(19) United States
(12) Patent Application Publication
(10) Pub. No.: US 2024/0051976 A1
(43) Pub. Date: Feb. 15, 2024

(54) A Tracer Compound and a Preparation Method Thereof

(71) Applicant: Turun Yliopisto, Turku (FI)

(72) Inventors: Anu AIRAKSINEN, Helsingin Yliopisto (FI); sofia OTARU, Helsingin Yliopisto (FI); Andreas PAULUS, Helsingin Yliopisto (FI); Mirrka SARPARANTA, Helsingin Yliopisto (FI)

(21) Appl. No.: 18/269,916

(22) PCT Filed: Dec. 23, 2021
(86) PCT No.: PCT/FI2021/050912

§ 371 Date: Jun. 27, 2023

(30) Foreign Application Priority Data
Dec. 28, 2020 (FI) 20206373

(51) Int. Cl. C07F 5/02; C07B 59/00

(52) U.S. Cl. CPC C07F 5/027; C07B 59/004; C07B 2200/05

(57) ABSTRACT

A tracer compound, or a pharmaceutically acceptable salt or solvate thereof is disclosed, the tracer compound having a structure comprising of a tetrazine moiety, a zwitterion moiety and a linker moiety, the linker moiety linking together the tetrazine moiety and the zwitterion moiety, and the linker moiety being comprised of specific parts S1-Y—S2 disclosed herein. Furthermore, an adduct of the tracer compound and a trans-cyclooctene (TCO)-derivatized targeting moiety, and a method for manufacturing the tracer compound and the adduct are disclosed.

### TECHNICAL FIELD

The present disclosure generally relates to radiolabeled radiopharmaceuticals. The disclosure relates particularly, though not exclusively, to radiolabeled radiopharmaceuticals obtained through inverse electron demand Diels-Alder cycloaddition reaction (IEDDA) of a trans-cyclooctene with a tetrazine moiety.

### BACKGROUND

This section illustrates useful background information without admission of any technique described herein representative of the state of the art.

Bioorthogonal chemistry has demonstrated great potential in synthesis of radiopharmaceuticals for diagnostic imaging. So far, the inverse electron demand Diels-Alder cycloaddition reaction (IEDDA) between tetrazines and dienophiles has shown the fastest reaction rate in bioorthogonal chemistry and it has been utilized in radiosynthesis of various fluorine-18 labeled radiopharmaceuticals, ranging from small molecules to macromolecules such as peptides and antibody fragments. The IEDDA reaction has been utilized also in pretargeted PET imaging using radionuclides such as ¹⁸F, ⁶⁸Ga and ⁶⁴Cu.

The inventors have surprisingly found out, that by synthesizing a compound comprising a tetrazine moiety and a specific zwitterion moiety and a linker moiety there between as described herein, a tracer compound with high modularity is obtained. Furthermore, an adduct comprising the tracer compound and a trans-cyclooctene (TCO)-derivatized targeting moiety, can be obtained through inverse electron demand Diels-Alder reaction (IEDDA) of a trans-cyclooctene with a tetrazine moiety of the tracer compound. The modularity of the linker moiety in the tracer compound enables use of the adduct and the tracer compound in targeting of plurality of different targeted entities in vivo and in vitro. The specific combination of the moieties comprised in the tracer compound and in the adduct provides a tracer compound with a good stability.

It is an object of the present disclosure to provide a tracer compound which as a part of the adduct exhibits high modularity in respect of targeting a number of different targeted entities in vivo and in vitro. It is also an object of the present disclosure to provide a tracer compound that has performance and/or stability which allows its use in applications of radiolabeling and radioimaging. Another object of the present disclosure is to provide a tracer compound with improved properties when used for targeting targeted entities in vivo and in vitro. Yet another object of the present disclosure is to provide an adduct of the tracer compound and the TCO-derivatized targeting moiety that can be used in targeting entities in vivo and in vitro.

### SUMMARY

The present application concerns the inventions defined in the appended independent claims, and their embodiments disclosed below. The appended claims define the scope of protection. Any method, process, product or apparatus disclosed in the description or drawing which is not covered by a claim is provided as an example which is not an embodiment of the claimed invention, but which is useful for understanding the claimed invention.

Herein is described a tracer compound and an adduct of the tracer compound with a trans-cyclooctene (TCO) derivatized targeting moiety, which through radiolabeling can be used in targeting of many diagnostic biomarkers in vivo and in vitro, and eventually detected through radioimaging methods. The inventors have surprisingly found, and shown in the examples below, that by combining of a tetrazine moiety, a specific linker moiety and a zwitterion moiety, a tracer compound can be synthesized, which has a fast synthesis and is highly modular in terms of targeting moieties that can be conjugated to it via a TCO moiety.

According to a first aspect is provided a tracer compound of formula (I), or a pharmaceutically acceptable salt or solvate thereof,

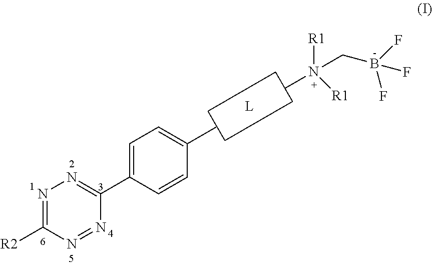

wherein:
each R1 is independently hydrogen (H) or an alkyl substituent with a formula CₙH₂ₙ₊₁, wherein n is an integer selected from the range 0-2; and
L is a linker moiety comprised of S1-Y—S2, wherein:
   Y is (—CH₂—)ₘ wherein m is an integer selected from the range 1-4, or Y is a polyethylene glycol linker -(PEG)ₓ-, wherein (PEG)ₓ contains x repetitive units of polyethylene oxide —CH₂—CH₂—O— groups, and x is an integer selected from the range 1-20; and
   S1 is —(CH₂)_{z}—CO—NH—(CH₂)_{z}—, or S1 is —(CH₂)_{z}—NH—CO—(CH₂)_{z}—, wherein each z is independently an integer selected from the range 0-4; and
   S2 is —CH₂—, or S2 is —(CH₂)_{f}—CO—NH—(CH₂)_{f}—, or S2 is —(CH₂)_{f}—NH—CO—(CH₂)_{f}—, wherein each f is independently an integer selected from the range 0-4; and
R2 is either hydrogen (H) or a phenyl substituent or an alkyl substituent with a formula CₛH₂ₛ₊₁, wherein s is an integer selected from the range 0-2.

According to a second aspect, there is provided an adduct of the tracer compound of the first aspect and a trans-cyclooctene (TCO)-derivatized targeting moiety, or a pharmaceutically acceptable salt or solvate thereof, obtained through inverse electron demand Diels-Alder reaction (IEDDA) of a TCO moiety of the TCO-derivatized targeting moiety, with a tetrazine moiety of the tracer compound.

According to a third aspect is provided the adduct of the second aspect for use in the detection of targeted entities in a subject by radioimaging, preferably by positron emission tomography imaging.

According to a fourth aspect is provided a method for manufacturing the tracer compound of the first aspect, the method comprising:
a. dissolving starting material into polar aprotic solvent and reacting the starting material with 2-(iodomethyl)-4,4,5,5-tetramethyl-1,3,2-dioxaborolane to provide an intermediate product; and
b. dissolving the intermediate product in polar aprotic solvent and reacting the intermediate product with KHF₂ in the presence of an acid such as HCl, water and organic solvent, to provide the tracer compound;

wherein the starting material is comprised of the tetrazine moiety linked via the linker moiety to a tertiary amine (—N(CH₃)₂); wherein
the tetrazine moiety is comprised of a 1,2,4,5-tetrazine ring, a phenyl ring attached to the C3 of the tetrazine ring, and R2 attached to the C6 of the tetrazine ring, wherein R2 is either hydrogen (H) or a phenyl substituent or an alkyl substituent with a formula CₛH₂ₛ₊₁, and s is an integer selected from the range 0-2;

and the linker moiety is comprised of S1-Y—S2, wherein:
Y is (—CH₂—)ₘ wherein m is an integer selected from the range 1-4, or Y is a polyethylene glycol linker -(PEG)ₓ-, wherein (PEG)ₓ contains x repetitive units of polyethylene oxide —CH₂—CH₂—O— groups, and x is an integer selected from the range 1-20; and
S1 is —(CH₂)_{z}—CO—NH—(CH₂)_{z}—, or S1 is —(CH₂)_{z}—NH—CO—(CH₂)_{z}—, wherein each z is independently an integer selected from the range 0-4; and
S2 is —CH₂—, or S2 is —(CH₂)_{f}—CO—NH—(CH₂)_{f}—, or S2 is —(CH₂)_{f}—NH—CO—(CH₂)_{f}—, wherein each f is independently an integer selected from the range 0-4.

According to a fifth aspect, there is provided a method for manufacturing the adduct of the second aspect or a pharmaceutically acceptable salt or solvate thereof, the method comprising:
providing the TCO-derivatized targeting moiety;
providing the tracer compound of the first aspect; and
allowing the tetrazine moiety of the tracer compound to react with the TCO moiety of the TCO-derivatized targeting moiety; and
radiolabeling the adduct with at least one ¹⁸F to obtain the adduct;

or the method comprising:
providing the TCO-derivatized targeting moiety;
providing the tracer compound of the first aspect;
radiolabeling the tracer compound of the first aspect with at least one ¹⁸F; and allowing the tetrazine moiety of the radiolabeled tracer compound to react with the TCO moiety of the TCO-derivatized targeting moiety to obtain the adduct.

According to a sixth aspect, there is provided a use of the tracer compound of the first aspect and/or the adduct of the second aspect, in detection of targeted entities in a subject by radioimaging the subject, wherein the entities are targeted with the radiolabeled tracer compounds and/or the adducts.

According to a seventh aspect, there is provided a kit production of the ¹⁸F-labeled adduct for detection of targeted entities in a subject with radioimaging, comprising at least one compartment containing the tracer compound of the first aspect, at least one compartment containing at least one TCO-derivatized targeting moiety, at least one compartment containing ¹⁸F for radiolabeling the tracer compound, and optionally aqueous and organic solvents for the IEDDA conjugation and radiolabeling of the tracer compound and/or the adduct.

According to a further aspect, there is provided a diagnostic and/or therapeutic use of the tracer compound of the first aspect and/or the adduct of the second aspect, in detection of targeted entities in a subject by radioimaging the subject, wherein the entities are targeted with the radiolabeled tracer compounds and/or the adducts.

According to a further aspect, there is provided a non-therapeutic use of the tracer compound of the first aspect and/or the adduct of the second aspect, in detection of targeted entities in a subject by radioimaging the subject, wherein the entities are targeted with the radiolabeled tracer compounds and/or the adducts. According to a further aspect, there is provided a non-diagnostic use of the tracer compound of the first aspect and/or the adduct of the second aspect, in detection of targeted entities in a subject by radioimaging the subject, wherein the entities are targeted with the radiolabeled tracer compounds and/or the adducts. In an embodiment, an example of a non-diagnostic and/or non-therapeutic use, is use in assessment of the structures to be targeted therapeutically.

The present tracer compound is advantageous in having high modularity due to the modular linker moiety, the linker moiety thereby providing an optimized conjugate with multiple different targeted entities. The present tracer compound of the first aspect is advantageous in having a low nonspecific tissue-uptake without the TCO-derivatized targeting moiety being conjugated to it. The present tracer compound of the first aspect is advantageous in pretargeted PET imaging.

The present adduct of the second aspect is advantageous as it allows a quick preparation of the readily usable adduct in room temperature. The present adduct of the second aspect is advantageous in being biocompatible for use in vivo. As shown in the examples provided below the adduct of the second aspect has a performance and specificity, which enable visualization of targeted entities through specific binding of a targeting moiety of an IEDDA cycloaddition product comprising the radiolabeled tracer compound.

The present adduct of the second aspect is advantageous in having a high target-tissue specific uptake rate and a low nonspecific tissue-uptake rate. The present radiolabeled adduct of the second aspect is advantageous in having a good metabolic stability and a fast clearance mainly through kidneys. The present adduct of the second aspect is advantageous in having a high modularity, the pharmacokinetics of the adduct being ductile through modification of the structural components of the tracer compound.

The present adduct of the second aspect is advantageous in having a high tumor-specific uptake rate, wherein the employed targeting moiety of the adduct targets biomolecules indicating cancerous growth.

### BRIEF DESCRIPTION OF THE FIGURES

Some example embodiments will be described with reference to the accompanying figures, in which:

FIG. 1 shows a synthesis route and a structural formula of a tracer compound AmBF₃-Tz (4) according to an example embodiment;

FIG. 2 shows a synthesis route and a structural formula of a tracer compound AmBF₃-PEG₄-Tz (8) according to an example embodiment;

FIG. 3 shows a synthesis route and a structural formula of a tracer compound AmBF₃-PEG₉-Tz (12) according to an example embodiment;

FIG. 4 shows a synthesis route and a structural formula of a trans-cyclooctene aldehyde (TCO—CHO) (15), and the structural formula of TCO-PEG₃-aldehyde (16), according to an example embodiment;

FIG. 5 shows a synthesis route and a structural formula of a PSMA-trans-cyclooctene (PSMA-TCO) (18), according to an example embodiment;

FIG. 6 shows a synthesis route and a structural formula of a PSMA-tranexamic acid-TCO (24), according to an example embodiment;

FIG. 7 shows a reaction for the radiolabeling of AmBF₃-Tz (4) prior to IEDDA conjugation resulting in ¹⁸F-labeled AmBF₃-Tz ([¹⁸F]4), according to an example embodiment;

FIG. 8 shows two alternative synthesis routes a) and b) for obtaining radiolabeled adduct which is an IEDDA cycloaddition product of the tracer compound AmBF₃ (4) and a TCO-derivatized targeting moiety, wherein the targeting moiety is represented as a peptide, according to an example embodiment;

FIG. 9 shows the % of total radioactivity observed at different timepoints as free, plasma membrane-bound or internalized radioactivity-%, after incubation of B16/F10 melanoma cells with [¹⁸F]AmBF₃-Tz ([¹⁸F]4). Over 99% of the total radioactivity is in the free fraction at each time point, thereby indicating a low unspecific binding of [¹⁸F]4 into plasma membranes.

FIG. 10 shows the % of radioactivity in the intracellular compartment of AR42J cells, from the total added radioactivity in the AR42J cell system, wherein the radioactivity was detected as a function of time, indicating a specific AR42J cell-uptake of TCO functionalized Tyr³-octreotide (TOC) conjugated with [¹⁸F]AmBF₃-Tz ([¹⁸F]4) forming [¹⁸F]AmBF₃-Tyr³-octreotide ([¹⁸F]25) in baseline conditions (Internalized). The uptake in baseline conditions was inhibited by co-incubation of the cells with [¹⁸F]25 together with non-modified blocking Octreotide (Blocked);

FIG. 11 shows a summation PET/CT image of a SCID mouse at 0-60 min post administration of [¹⁸F]AmBF₃-Tz ([¹⁸F]4), B.=bladder, G.B.=gallbladder, K.=kidney, L.=liver;

FIG. 12 a shows the percentage of injected dose per gram of tissue (% ID/g) quantifying radioactivity in the indicated tissues (a), and in urine and feces (b), at 270±2 minutes after intravenous administration of [¹⁸F]AmBF₃-Tz ([¹⁸F]4) in control SCID and C57BL/6JRj mice (n=2-3/strain), indicating the clearance and excretion of the radioactive tracer compound, respectively;

FIG. 13 shows the standardized uptake value (SUV) measured in indicated tissues (a), and urine (b), during 60 min following the administration of [¹⁸F]AmBF₃-Tz ([¹⁸F]4) in a male SCID mouse (n=1), indicating the biodistribution and elimination of [¹⁸F]4;

FIG. 14 shows the percentage of injected dose per gram of tissue (% ID/g) of [¹⁸F]25 measured in different tissues of AR42J-tumor bearing Rj:NMRI-Foxn1 nu/nu mice (n=2-4) after intravenous administration of radiolabeled adduct [¹⁸F]25, indicating its biodistribution at various time points;

FIG. 15 shows a summation PET/CT image of AR42J tumor bearing Rj:NMRI-Foxn1 nu/nu mice at 20-80 min post administration of [¹⁸F]AmBF₃-Tyr³-octreotide ([¹⁸F]25). The animal on the left was co-administered with non-modified blocking octreotide and [¹⁸F]25 intravenously. The animal on the right was administered only with [¹⁸F]25 without the blocking octreotide, enabling the visualization of the subcutaneous AR42J tumor on the right shoulder, T=tumor;

FIG. 16 shows the standardized uptake value (SUV) measured in AR42J tumor tissue as a function of time (min). Here, the AR42J tumor bearing mice (n=2/group) were administered with [¹⁸F]AmBF₃-Tyr³-octreotide ([¹⁸F]25) alone (Non-blocked) or co-administered with [¹⁸F]25 and blocking octreotide (Blocked) intravenously;

FIG. 17 shows the standardized uptake value (SUV) measured in indicated tissues as a function of time (min), indicating the clearance of radioactivity after intravenous administration of [¹⁸F]AmBF₃-Tyr³-octreotide ([¹⁸F]25) in a tumor bearing AR42J mouse (n=1);

FIG. 18 shows maximum intensity projections of PET/CT at 15-90 min post-injection of [¹⁸F]AmBF₃-tranexamic acid-PSMA ([¹⁸F]29). The animal on the right was co-administered with 2-PMPA (blocking) and [¹⁸F]29 intravenously. The animal on the left was administered only with [¹⁸F]29 without the blocking 2-PMPA, enabling the visualization of the subcutaneous C4-2 tumor on the left shoulder, T=tumor, G=gallbladder, K=kidney, U=bladder/urine;

FIG. 19 shows the percentage of injected dose per gram of tissue (% ID/g) of [¹⁸F]29 measured in different tissues of C4-2-tumor bearing SCID mice (n=3) after intravenous administration of radiolabeled adduct [¹⁸F]29, indicating its biodistribution at 60 min. post injection.

### DETAILED DESCRIPTION

As used herein, the term “tracer compound” or “tracer” means a chemical compound that can be traced with radiation detectors. In an embodiment, the tracer compound contains one or more atoms that have been replaced by a radionuclide. In an embodiment, the tracer compound is the tracer compound according to the first aspect.

As used herein, the term “IEDDA” means inverse electron demand Diels-Alder reaction. As used herein, the term “moiety” means a part of a molecule, which can be functionally or structurally identified in the structure of the molecule, for example, the tracer compound or the adduct as a whole. A moiety can thus be named individually.

As used herein, the term “linker” or “linker moiety” means a modular region connecting two adjacent moieties within the tracer compound or within the adduct. In an embodiment, by linker moiety is meant the linker moiety according to the first aspect, linking together the tetrazine moiety and the zwitterion moiety of the tracer compound. In an embodiment, the S2 of the linker moiety is attached to the N of the zwitterion moiety, and the S1 of the linker moiety is attached to the phenyl of the tetrazine moiety of the tracer compound. In an alternative embodiment, “linker”, or “linking moiety” or “linker moiety” means the linker moiety in the TCO derivatized targeting moiety, linking together the targeting moiety and the TCO moiety.

As used herein, the term “tetrazine” means a six-membered aromatic tetrazine ring containing four nitrogen atoms. As used herein, the term tetrazine refers to 1,2,4,5-tetrazine isomer.

As used herein, the term “tetrazine moiety” means a moiety comprising a six-membered aromatic ring, which comprises four nitrogen atoms. The tetrazine structure in the tetrazine moiety is a 1,2,4,5-tetrazine isomer structure. The tetrazine moiety further comprises a six-membered aromatic phenyl ring, which is attached to the C3 of the tetrazine ring. The tetrazine moiety further comprises a R2 attached to the C6 of the tetrazine ring, wherein the R2 is either hydrogen (H) or a phenyl substituent or an alkyl substituent with a formula CₛH₂ₛ₊₁, wherein s is an integer selected from the range 0-2. In an embodiment, the linker moiety L of the tracer compound according to formula (I), and the 1,2,4,5-tetrazine of the tetrazine moiety, are attached to the phenyl ring of the tetrazine moiety in para positions with respect to each other, according to the formula (I).

As used herein, the term “zwitterion” denotes a molecule that has at least two functional groups and contains an equal number of positively and negatively charged functional groups. The overall charge of a zwitterion molecule is zero. In an embodiment, the zwitterion is an organotrifluoroborate [ABF₃]⁻ according to formula (I), wherein the A is [—CH₂—N—(R1)₂]⁺.

As used herein, the term “alkyl substituent” means in context of chemical structural formulas a generic (unspecified) alkane which is a part of another molecule and which is missing one hydrogen. The smallest “alkyl substituent” is a methyl group, with the formula CH₃—.

As used herein, the term “polyethylene glycol linker (-(PEG)ₓ-) means a modular moiety connecting two other adjacent moieties within the tracer compound or within the adduct, wherein (PEG)ₓ contains x repetitive units of polyethylene oxide —CH₂—CH₂—O— groups, and x is an integer. The (PEG)ₓ moiety of the linker may be surrounded by separate linking moieties, referred here as S1 and S2, linking the linker moiety with the rest of the tracer compound or the adduct.

As used herein, the term “integer” means a whole number, not a fractional number, that can be positive, negative, or zero.

As used herein, the term “¹⁸F” or “Fluorine-18” means a fluorine radioisotope which decays mainly by positron emission.

As used herein, the term “adduct” refers to an adduct product obtained through addition of two or more distinct molecules, resulting in a single product. In an embodiment the term “adduct” refers to an adduct of the tracer compound and a TCO-derivatized targeting moiety.

As used herein, the term “trans-cyclooctene (TCO)” refers to a trans-isomer of a cycloalkene with a chain of eight carbons forming the cyclic hydrocarbon, wherein two C—C single bonds on both sides of a C═C double bond are on opposite sides of the latter's plane.

As used herein, the term “TCO moiety” refers to a TCO, which is part of a molecule comprising at least one other moiety, such as a targeting moiety or linking moiety, linked to the said TCO. As used herein, the term “TCO moiety” refers to the trans-isomer of the cyclic TCO of the TCO derivatized targeting moiety prior to IEDDA conjugation.

As used herein, the term “TCO-derivatized targeting moiety” means a targeting moiety, which is derivatized with a trans-cyclooctene (TCO) moiety. In an embodiment, the TCO-derivatized targeting moiety comprises a linking moiety between the targeting moiety and the TCO moiety.

As used herein, the term “tertiary amine” refers to a compound comprising of carbon, hydrogen, and nitrogen atoms, wherein an amine nitrogen has three carbons attached to it, constituting three organic substituents. In an embodiment, tertiary amine is N—(K)₃, wherein each K is independently an alkyl or an aryl. In an embodiment, tertiary amine is (—N(CH₃)₂), wherein the nitrogen is attached to a third carbon which is part of another moiety of the compound the tertiary amine is part of. In an embodiment, tertiary amine is (—N(CH₃)₂), wherein the nitrogen is attached to a third carbon which is part of the linker moiety of the tracer compound.

As used herein, with the term “targeting moiety” is meant a peptide, an antibody, an antibody fragment, or a nanoparticle, which targets through its sequence and/or 3D (surface) structure a desired targeted entity. In an embodiment, the targeting moiety is part of a larger structure or a compound and is able to guide or co-locate the said compound to respective targeted entities in vitro as well as in vivo.

As used herein, the term “targeted entity” refers to a sequence and/or 3D (surface) structure, which is targeted by the targeting moiety i.e. of the adduct in vitro and/or in vivo, and recognized through its sequence and/or 3D (surface) structure thereby resulting in co-location of the targeting moiety with the targeted entity. An example of a targeted entity is a biomolecule.

As used herein, a “peptide” is an amino acid sequence including a plurality of consecutive polymerized amino acid residues. For purpose of this disclosure, peptides are molecules including up to 50 amino acid residues. The peptide may include modified amino acid residues, naturally occurring amino acid residues not encoded by a codon, and non-naturally occurring amino acid residues.

As used herein, the term “antibody” means an immunoglobulin protein that recognizes the epitope of an antigen via its fragment antigen-binding (Fab) variable region and binds to it.

As used herein, the term “radioimaging” refers to a method employing radioactive substances to visualize and measure physiological structures and activities inside macro- or micro-organisms.

As used herein, the term “Positron Emission Tomography imaging (PET)” means an imaging technique using radiotracers to visualize and measure physiological structures and activities, utilizing a medical scintillography technique and detection of gamma rays by gamma cameras. As used herein, the term “positron emission tomography imaging (PET)” comprises also the Positron Emission Tomography-computed tomography (PET-CT), further integrating an x-ray computed tomography (CT) scanner for sequential image acquisition thereby forming a combined single superposed (co-registered) image.

As used herein, the term “pretargeted PET imaging” means a two-step PET labelling process, wherein a targeted entity is first targeted and conjugated with a targeting moiety, without the tracer compound attached to it. This is followed by a second step, wherein the radiolabeled tracer compound is delivered in contact with the targeting moiety and allowed to bind to it. In an embodiment, the targeting moiety is a TCO-derivatized targeting moiety and the binding of the TCO-derivatized targeting moiety to the tracer compound occurs through IEDDA conjugation.

As used herein, the term “target-tissue specific uptake” means the uptake or binding of the tracer compound or the adduct to a target tissue of interest, through the binding of the targeting moiety of the adduct to a specific targeted entity in the target-tissue.

As used herein, the term “tumor-specific uptake” means the uptake or binding of the tracer compound or the adduct to the target tissue of interest, through the binding of the targeting moiety of the adduct to the targeted entity in the target-tissue, wherein the target-tissue is cancerous tissue.

As used herein, the term “comprising” includes the broader meanings of “including”, “containing”, and “comprehending”, as well as the narrower expressions “consisting of” and “consisting only of”.

The term “fluorination” refers to a chemical reaction by which fluorine is introduced into a compound. In an embodiment, the fluorine is a stable isotope fluorine-19 (¹⁹F). In another embodiment, the fluorine is a radioisotope ¹⁸F.

As used herein, the term “organotrifluoroborate” means an organoboron compound with the general molecular formula [ABF₃]⁻. The “A” in the organotrifluoroborate general formula can be a positively charged functional group A⁺, making the organotrifluoroborate moiety a zwitterion.

As used herein, the term “[¹⁸F]trifluoroborate” means an organotrifluoroborate, wherein at least one of the three fluoride atoms [F], is replaced with ¹⁸F-fluoride isotope.

As used herein, the term “biomolecule” refers to any molecule of medical, physiological or scientific significance, analog or derivative thereof that may be compatible with a biological system or which may or may not possess biological activity.

As used herein, the term “antibody fragment” means a piece of an entire antibody molecule, such as an antigen-binding fragment of an antibody molecule (Fab) or a crystallizable fragment of an antibody molecule (Fc, the tail region).

As used herein, the term nanoparticle means an article of matter of any shape that has dimension(s) between 1 and 300 nanometers (nm) in diameter. As an example, a nanoparticle is an organic nanocrystal, an inorganic nanocrystal, or a liposome.

As used herein, —(CH₂)₀— means no CH₂ is present at the indicated position, —(CH₂)₁— means —CH₂—, —(CH₂)₂— means —CH₂—CH₂—, —(CH₂)₃— means —CH₂—CH₂—CH₂—, —(CH₂)₄— means —CH₂—CH₂—CH₂—CH₂—.

As used herein, the term “Tyr³-octreotide” means an octapeptide, i.e., an oligopeptide having eight amino acids, wherein a phenylalanine at the 3^{rd} position of the octreotide is substituted with a tyrosine. Both octreotide and Tyr³-octreotide are able to mimic natural somatostatin pharmacologically and to bind to somatostatin receptors, which are overexpressed in neuroendocrine tumors. Octreotide or Tyr³-octreotide may be used as a targeting moiety or as a model of a targeting moiety for somatostatin receptors.

As used herein, the term “PSMA” means a prostate-specific membrane antigen, which is a type II membrane glycoprotein, and which is over expressed in prostate cancer. In context of the compounds 28 and 29 disclosed in this application, the term “PSMA” refers to a prostate-specific membrane antigen (PSMA) targeting moiety, which can function as a ligand binding to PSMA. Thereby, the compounds 28 and 29 comprise a targeting moiety or a model of a targeting moiety for prostate-specific membrane antigen.

As used herein, the term “tautomer” refers to either of at least two structural isomers of a chemical compound, which can exist simultaneously and are readily interchangeable by migration of an atom or group within the molecule.

As used herein, the term “non-therapeutic use” refers to a use which is not aimed to any therapeutic aspect of a disease management. In an embodiment, the term “non-therapeutic use” can refer to a use for diagnostic purposes. As used herein, the term “non-diagnostic use” refers to a use which is not aimed to diagnosing a disease.

In an embodiment, the tracer compound comprises a zwitterion moiety, a linker moiety and a tetrazine moiety. In certain other embodiments, the tracer compound comprises also other moieties or side groups. In an embodiment, the linker moiety is positioned between the zwitterion moiety and the tetrazine moiety.

In an embodiment, the zwitterion moiety of the tracer compound comprises an organotrifluoroborate. The organotrifluoroborate comprises a (BF₃)⁻ moiety linked to a positively (+) charged (cationic) group. More specifically, the (BF₃)⁻ group is linked through —CH₂ to a positively (+) charged (cationic) group (N(R1)₂)⁺.

In an embodiment, each R1 of the tracer compound is independently an alkyl substituent with a formula CₙH₂ₙ₊₁, wherein n is an integer selected from the range 0-2. In an embodiment each R1 is independently an alkyl substituent with a carbon chain length of C2 or less. In an embodiment, each R1 of the tracer compound is independently an alkyl substituent with a formula C₁H₃. In an embodiment, each R1 of the tracer compound is independently an alkyl substituent with a formula C₂H₅. In an embodiment, each R1 is independently hydrogen (H). In an embodiment, each R1 is independently a methyl group. In an embodiment, each R1 is independently an ethyl group.

In an embodiment, each alkyl substituent R1 allows individually the nucleophilic attack on the carbon between the nitrogen (N) and boron (B) within the zwitterion moiety. In an embodiment, each alkyl substituent R1 is a non-interfering group with regard to fluorination of the Boron (B). Non-interfering in this context means that the R1 does not fully or substantially prevent fluorination of the Boron (B).

In an embodiment, the linker moiety is comprised of units S1-Y—S2, wherein Y represents the core unit structure of the linker, and S1 and S2 represent the chains on both sides of the core unit Y, linking the linker moiety on the S1 side to the tetrazine moiety, and on the S2 side to the zwitterion moiety.

In an embodiment, Y of the tracer compound is (—CH₂—)ₘ, wherein m is 1, 2, 3, or 4. In an embodiment, m is an integer <5.

In an embodiment, Y of the linker moiety is (—CH₂—)ₘ, S1 is —(CH₂)_{z}—CO—NH—(CH₂)_{z}—, and S2 is —CH₂, wherein m is an integer selected from the range 1-4, each z is independently an integer selected from the range 0-4, each R1 of the tracer compound is independently an alkyl substituent with a formula CₙH₂ₙ₊₁ wherein n is an integer selected from the range 0-2 or hydrogen (H), and the R2 of the tracer compound is either hydrogen (H) or a phenyl substituent or an alkyl substituent with a formula CₛH₂ₛ₊₁, wherein s is an integer selected from the range 0-2.

In an embodiment, Y of the linker moiety is (—CH₂—)ₘ, S1 is —(CH₂)_{z}—CO—NH—(CH₂)_{z}—, and S2 is —(CH₂)_{f}—CO—NH—(CH₂)_{f}—, wherein m is an integer selected from the range 1-4, each z and f is independently an integer selected from the range 0-4, each R1 of the tracer compound is independently H or an alkyl substituent with a formula CₙH₂ₙ₊₁ wherein n is an integer selected from the range 0-2, and the R2 of the tracer compound is either H or a phenyl substituent or an alkyl substituent with a formula CₛH₂ₛ₊₁, wherein s is an integer selected from the range 0-2.

In an embodiment, Y of the linker moiety is (—CH₂—)ₘ, S1 is —(CH₂)_{z}—CO—NH—(CH₂)_{z}—, and S2 is —(CH₂)_{f}—NH—CO—(CH₂)_{f}—, wherein m is an integer selected from the range 1-4, each z and f is independently an integer selected from the range 0-4, each R1 of the tracer compound is independently H or an alkyl substituent with a formula CₙH₂ₙ₊₁ wherein n is an integer selected from the range 0-2, and the R2 of the tracer compound is either H or a phenyl substituent or an alkyl substituent with a formula CₛH₂ₛ₊₁, wherein s is an integer selected from the range 0-2.

In an embodiment, Y of the linker moiety is (—CH₂—)ₘ, S1 is —(CH₂)_{z}—NH—CO—(CH₂)_{z}—, and S2 is —CH₂—, wherein m is an integer selected from the range 1-4, each z is independently an integer selected from the range 0-4, each R1 of the tracer compound is independently H or an alkyl substituent with a formula CₙH₂ₙ₊₁ wherein n is an integer selected from the range 0-2, and the R2 of the tracer compound is either H or a phenyl substituent or an alkyl substituent with a formula CₛH₂ₛ₊₁, wherein s is an integer selected from the range 0-2.

In an embodiment, Y of the linker moiety is (—CH₂—)ₘ, S1 is —(CH₂)_{z}—NH—CO—(CH₂)_{z}—, and S2 is —(CH₂)_{f}—CO—NH—(CH₂)_{f}—, wherein m is an integer selected from the range 1-4, each z and f is independently an integer selected from the range 0-4, each R1 of the tracer compound is independently H or an alkyl substituent with a formula CₙH₂ₙ₊₁ wherein n is an integer selected from the range 0-2, and the R2 of the tracer compound is either H or a phenyl substituent or an alkyl substituent with a formula CₛH₂ₛ₊₁, wherein s is an integer selected from the range 0-2.

In an embodiment, Y of the linker moiety is (—CH₂—)ₘ, S1 is —(CH₂)_{z}—NH—CO—(CH₂)_{z}, and S2 is —(CH₂)_{f}—NH—CO—(CH₂)_{f}—, wherein m is an integer selected from the range 1-4, each z and f is independently an integer selected from the range 0-4, each R1 of the tracer compound is independently H or an alkyl substituent with a formula CₙH₂ₙ₊₁ wherein n is an integer selected from the range 0-2, and the R2 of the tracer compound is either H or a phenyl substituent or an alkyl substituent with a formula CₛH₂ₛ₊₁, wherein s is an integer selected from the range 0-2.

In an embodiment, Y of the linker moiety is (—CH₂—)ₘ, S1 is —(CH₂)₁—CO—NH—(CH₂)₀—, and S2 is —CH₂, wherein m is an integer selected from the range 1-4. In an embodiment, Y is (—CH₂—)₁, S1 is —(CH₂)₁—CO—NH—(CH₂)₀—, and S2 is —CH₂. In an embodiment, Y is (—CH₂—)₂ the S1 is —(CH₂)₁—CO—NH—(CH₂)₀—, and the S2 is —CH₂—. In an embodiment, Y is (—CH₂—)₃, the S1 is —(CH₂)₁—CO—NH—(CH₂)₀— and the S2 is —CH₂—. In an embodiment, Y is (—CH₂—)₄, the S1 is —(CH₂)₁—CO—NH—(CH₂)₀—, and the S2 is —CH₂—. In an embodiment, Y of the linker moiety is (—CH₂—)₁, S1 is —(CH₂)₁—CO—NH—(CH₂)₀—, S2 is —CH₂, both R1 are independently CH₃, and R2 is H.

In an embodiment, Y of the linker moiety is (—CH₂—)ₘ, S1 is —(CH₂)₁—NH—CO—(CH₂)₀—, and S2 is —CH₂, wherein m is an integer selected from the range 1-4. In an embodiment, Y is (—CH₂—)₁, S1 is —(CH₂)₁—NH—CO—(CH₂)₀—, and S2 is —CH₂. In an embodiment, Y is (—CH₂—)₂ the S1 is —(CH₂)₁—NH—CO—(CH₂)₀—, and S2 is —CH₂—. In an embodiment, Y is (—CH₂—)₃, S1 is —(CH₂)₁—NH—CO—(CH₂)₀— and S2 is —CH₂—. In an embodiment, Y is (—CH₂—)₄, S1 is —(CH₂)₁—NH—CO—(CH₂)₀—, and S2 is —CH₂—.

In an embodiment, Y of the linker moiety is -(PEG)ₓ-, S1 is —(CH₂)_{z}—CO—NH—(CH₂)_{z}—, and S2 is —CH₂—, wherein x is an integer selected from the range 1-20, each z is independently an integer selected from the range 0-4, each R1 of the tracer compound is independently H or an alkyl substituent with a formula CₙH₂ₙ₊₁ wherein n is an integer selected from the range 0-2, and the R2 of the tracer compound is either H or a phenyl substituent or an alkyl substituent with a formula CₛH₂ₛ₊₁, wherein s is an integer selected from the range 0-2.

In an embodiment, Y of the linker moiety is -(PEG)ₓ-, S1 is —(CH₂)_{z}—CO—NH—(CH₂)_{z}—, and S2 is —(CH₂)_{f}—CO—NH—(CH₂)_{f}, wherein x is an integer selected from the range 1-20, each z and f is independently an integer selected from the range 0-4, each R1 of the tracer compound is independently H or an alkyl substituent with a formula CₙH₂ₙ₊₁ wherein n is an integer selected from the range 0-2, and the R2 of the tracer compound is either H or a phenyl substituent or an alkyl substituent with a formula CₛH₂ₛ₊₁, wherein s is an integer selected from the range 0-2.

In an embodiment, Y of the linker moiety is -(PEG)ₓ-, S1 is —(CH₂)_{z}—CO—NH—(CH₂)_{z}—, and S2 is —(CH₂)_{f}—NH—CO—(CH₂)_{f}, wherein x is an integer selected from the range 1-20, each z and f is dependently an integer selected from the range 0-4, each R1 of the tracer compound is independently H or an alkyl substituent with a formula CₙH₂ₙ₊₁ wherein n is an integer selected from the range 0-2, and the R2 of the tracer compound is either H or a phenyl substituent or an alkyl substituent with a formula CₛH₂ₛ₊₁, wherein s is an integer selected from the range 0-2.

In an embodiment, Y of the linker moiety is -(PEG)ₓ-, S1 is —(CH₂)_{z}—NH—CO—(CH₂)_{z}—, and S2 is —CH₂—, wherein x is an integer selected from the range 1-20, each z is independently an integer selected from the range 0-4, each R1 of the tracer compound is independently H or an alkyl substituent with a formula CₙH₂ₙ₊₁ wherein n is an integer selected from the range 0-2, and the R2 of the tracer compound is either H or a phenyl substituent or an alkyl substituent with a formula CₛH₂ₛ₊₁, wherein s is an integer selected from the range 0-2.

In an embodiment, Y of the linker moiety is -(PEG)ₓ-, S1 is —(CH₂)_{z}—NH—CO—(CH₂)_{z}—, and S2 is —(CH₂)_{f}—CO—NH—(CH₂)_{f}, wherein x is an integer selected from the range 1-20, each z and f is independently an integer selected from the range 0-4, each R1 of the tracer compound is independently H or an alkyl substituent with a formula CₙH₂ₙ₊₁ wherein n is an integer selected from the range 0-2, and the R2 of the tracer compound is either H or a phenyl substituent or an alkyl substituent with a formula CₛH₂ₛ₊₁, wherein s is an integer selected from the range 0-2.

In an embodiment, Y of the linker moiety is -(PEG)ₓ-, S1 is —(CH₂)_{z}—NH—CO—(CH₂)_{z}—, and S2 is —(CH₂)_{f}—NH—CO—(CH₂)_{f}, wherein x is an integer selected from the range 1-20, each z and f is independently an integer selected from the range 0-4, each R1 of the tracer compound is independently H or an alkyl substituent with a formula CₙH₂ₙ₊₁ wherein n is an integer selected from the range 0-2, and the R2 of the tracer compound is either H or a phenyl substituent or an alkyl substituent with a formula CₛH₂ₛ₊₁, wherein s is an integer selected from the range 0-2.

In an embodiment is provided a tracer compound according to formula (I) or a pharmaceutically acceptable salt or solvate thereof, wherein: each R1 is independently hydrogen (H) or an alkyl substituent with a formula CₙH₂ₙ₊₁, wherein n is an integer selected from the range 0-2; L is a linker moiety comprised of S1-Y—S2, wherein:
Y is (—CH₂—)ₘ wherein m is an integer selected from the range 1-4, or Y is a polyethylene glycol linker -(PEG)ₓ-, wherein (PEG)ₓ contains x repetitive units of polyethylene oxide —CH₂—CH₂—O— groups, and x is an integer selected from the range 1-20; and
   S1 is —CH₂—NH—CO—(CH₂)_{z}—, or —CH₂—CO—NH—(CH₂)_{z}—, wherein each z is independently an integer selected from the range 0-4; and
S2 is —(CH₂)_{f}, or —(CH₂)_{f}—CO—NH—CH₂—CH₂—, or —(CH₂)_{f}—NH—CO—CH₂—CH₂—, wherein each f is independently an integer selected from the range 0-4; and
R2 is either hydrogen (H) or a phenyl substituent or an alkyl substituent with a formula CₛH₂ₛ₊₁, wherein s is an integer selected from the range 0-2.

In an embodiment is provided a tracer compound according to formula (I) or a pharmaceutically acceptable salt or solvate thereof, wherein:
each R1 is independently hydrogen (H) or an alkyl substituent with a formula CₙH₂ₙ₊₁, wherein n is an integer selected from the range 0-2; and
L is a linker moiety comprised of S1-Y—S2, wherein:
   Y is (—CH₂—)ₘ wherein m is an integer selected from the range 1-4, S1 is —CH₂—CO—NH—, or —CH₂—NH—CO— and S2 is —CH₂—; or
   Y is a polyethylene glycol linker -(PEG)ₓ-, wherein (PEG)ₓ contains x repetitive units of polyethylene oxide —CH₂—CH₂—O— groups, and x is an integer selected from the range 1-20; and
   S1 is —CH₂—NH—CO—(CH₂)_{z}—, or —CH₂—CO—NH—(CH₂)_{z}—, wherein each z is independently an integer selected from the range 0-4; and
   S2 is —(CH₂)_{f}—CO—NH—CH₂—CH₂— or —(CH₂)_{f}—NH—CO—CH₂—CH₂—, wherein each f is independently an integer selected from the range 0-4; and
R2 is either hydrogen (H) or a phenyl substituent or an alkyl substituent with a formula CₛH₂ₛ₊₁, wherein s is an integer selected from the range 0-2.

In an embodiment, Y is a polyethylene glycol linker -(PEG)ₓ-, wherein (PEG)ₓ contains x repetitive units of polyethylene oxide —CH₂—CH₂—O— groups, and x is an integer selected from the range 1-20. In an embodiment, the x in -(PEG)ₓ- of the tracer compound is an integer selected from the range 1-15 or from the range 1-10. In an embodiment, x in -(PEG)ₓ- is an integer selected from the range 1-9, or from the range 1-8, or from the range 1-7, or from the range 1-6, or from the range 1-5, or from the range 1-4, or from the range 1-3, or from the range 1-2, or x is 1. In an embodiment, x in -(PEG)ₓ- of the tracer compound is 4 or 9. In an embodiment, x in -(PEG)ₓ- of the tracer compound is 1, 2, 3, 4, 5, 6, 7, 8, 9, 10, 11, 12, 13, 14, 15, 16, 17, 18, 19 or 20.

In an embodiment, Y is (—CH₂—)ₘ, S1 is —CH₂—CO—NH— and S2 is —CH₂—, wherein m is an integer selected from the range 1-4, each R1 of the tracer compound is independently H or an alkyl substituent with a formula CₙH₂ₙ₊₁ wherein n is an integer selected from the range 0-2, and the R2 of the tracer compound is either H or a phenyl substituent or an alkyl substituent with a formula CₛH₂ₛ₊₁, wherein s is an integer selected from the range 0-2.

In an embodiment, Y is (—CH₂—)ₘ, S1 is —CH₂—NH—CO—, and S2 is —CH₂—, wherein m is an integer selected from the range 1-4, each R1 of the tracer compound is independently H or an alkyl substituent with a formula CₙH₂ₙ₊₁ wherein n is an integer selected from the range 0-2, and the R2 of the tracer compound is either H or a phenyl substituent or an alkyl substituent with a formula CₛH₂ₛ₊₁, wherein s is an integer selected from the range 0-2.

In an embodiment, Y is (—CH₂—)₁, S1 is —CH₂—CO—NH— or —CH₂—NH—CO—, S2 is —CH₂—, each R1 is independently H or an alkyl substituent with a formula CₙH₂ₙ₊₁ wherein n is an integer selected from the range 0-2, and R2 is a phenyl substituent.

In an embodiment, Y is (—CH₂—)₁, S1 is —CH₂—CO—NH— or —CH₂—NH—CO—, S2 is —CH₂—, each R1 is independently H or an alkyl substituent with a formula CₙH₂ₙ₊₁ wherein n is an integer selected from the range 0-2, and R2 is H.

In an embodiment, Y is (—CH₂—)₁, S1 is —CH₂—CO—NH— or —CH₂—NH—CO—, S2 is —CH₂—, each R1 is independently H or an alkyl substituent with a formula CₙH₂ₙ₊₁ wherein n is an integer selected from the range 0-2, and R2 is an alkyl substituent with a formula C₁H₃.

In an embodiment, Y is (—CH₂—)₁, S1 is —CH₂—CO—NH— or —CH₂—NH—CO—, S2 is —CH₂—, each R1 is independently H or an alkyl substituent with a formula CₙH₂ₙ₊₁ wherein n is an integer selected from the range 0-2, and R2 is an alkyl substituent with a formula C₂H.

In an embodiment, Y is (—CH₂—)₂, S1 is —CH₂—CO—NH— or —CH₂—NH—CO—, S2 is —CH₂—, each R1 of the tracer compound is independently H or an alkyl substituent with a formula CₙH₂ₙ₊₁ wherein n is an integer selected from the range 0-2, and R2 is a phenyl substituent.

In an embodiment, Y is (—CH₂—)₂, S1 is —CH₂—CO—NH— or —CH₂—NH—CO—, S2 is —CH₂—, each R1 of the tracer compound is independently H or an alkyl substituent with a formula CₙH₂ₙ₊₁ wherein n is an integer selected from the range 0-2, and R2 is H.

In an embodiment, Y is (—CH₂—)₂, S1 is —CH₂—CO—NH— or —CH₂—NH—CO—, S2 is —CH₂—, each R1 of the tracer compound is independently H or an alkyl substituent with a formula CₙH₂ₙ₊₁ wherein n is an integer selected from the range 0-2, and R2 is an alkyl substituent with a formula C₁H₃.

In an embodiment, Y is (—CH₂—)₂, S1 is —CH₂—CO—NH— or —CH₂—NH—CO—, S2 is —CH₂—, each R1 of the tracer compound is independently H or an alkyl substituent with a formula CₙH₂ₙ₊₁ wherein n is an integer selected from the range 0-2, and R2 is an alkyl substituent with a formula C₂H.

In an embodiment, Y is (—CH₂—)₃, S1 is —CH₂—CO—NH— or —CH₂—NH—CO—, S2 is —CH₂—, each R1 of the tracer compound is independently H or an alkyl substituent with a formula CₙH₂ₙ₊₁ wherein n is an integer selected from the range 0-2, and R2 is a phenyl substituent.

In an embodiment, Y is (—CH₂—)₃, S1 is —CH₂—CO—NH— or —CH₂—NH—CO—, S2 is —CH₂—, each R1 of the tracer compound is independently H or an alkyl substituent with a formula CₙH₂ₙ₊₁ wherein n is an integer selected from the range 0-2, and R2 is H.

In an embodiment, Y is (—CH₂—)₃, S1 is —CH₂—CO—NH— or —CH₂—NH—CO—, S2 is —CH₂—, each R1 of the tracer compound is independently H or an alkyl substituent with a formula CₙH₂ₙ₊₁ wherein n is an integer selected from the range 0-2, and R2 is an alkyl substituent with a formula C₁H₃.

In an embodiment, Y is (—CH₂—)₃, S1 is —CH₂—CO—NH— or —CH₂—NH—CO—, S2 is —CH₂—, each R1 of the tracer compound is independently H or an alkyl substituent with a formula CₙH₂ₙ₊₁ wherein n is an integer selected from the range 0-2, and R2 is an alkyl substituent with a formula C₂H.

In an embodiment, Y is (—CH₂—)₄, S1 is —CH₂—CO—NH— or —CH₂—NH—CO—, S2 is —CH₂—, each R1 of the tracer compound is independently H or an alkyl substituent with a formula CₙH₂ₙ₊₁ wherein n is an integer selected from the range 0-2, and R2 is a phenyl substituent.

In an embodiment, Y is (—CH₂—)₄, S1 is —CH₂—CO—NH— or —CH₂—NH—CO—, S2 is —CH₂—, each R1 of the tracer compound is independently H or an alkyl substituent with a formula CₙH₂ₙ₊₁ wherein n is an integer selected from the range 0-2, and R2 is H.

In an embodiment, Y is (—CH₂—)₄, S1 is —CH₂—CO—NH— or —CH₂—NH—CO—, S2 is —CH₂—, each R1 of the tracer compound is independently H or an alkyl substituent with a formula CₙH₂ₙ₊₁ wherein n is an integer selected from the range 0-2, and R2 is an alkyl substituent with a formula C₁H₃.

In an embodiment, Y is (—CH₂—)₄, S1 is —CH₂—CO—NH— or —CH₂—NH—CO—, S2 is —CH₂—, each R1 of the tracer compound is independently H or an alkyl substituent with a formula CₙH₂ₙ₊₁ wherein n is an integer selected from the range 0-2, and R2 is an alkyl substituent with a formula C₂H₅.

In an embodiment, each R1 of the tracer compound is independently C₁H₃, Y is (—CH₂—)₁, S1 is —CH₂—CO—NH—, S2 is —CH₂—, and R2 is a H.

In an embodiment, Y is -(PEG)ₓ-, S1 is —CH₂—NH—CO—(CH₂)₀—, and S2 is —(CH₂)₀—CO—NH—CH₂—CH₂—. In an embodiment, Y is -(PEG)ₓ-, S1 is —CH₂—NH—CO—(CH₂)₁—, and S2 is —(CH₂)₀—CO—NH—CH₂—CH₂—. In an embodiment, Y is -(PEG)ₓ-, S1 is —CH₂—NH—CO—(CH₂)₂—, and S2 is —(CH₂)₀—CO—NH—CH₂—CH₂—. In an embodiment, Y is -(PEG)ₓ-, S1 is —CH₂—NH— CO—(CH₂)₃—, and S2 is —(CH₂)₀—CO—NH—CH₂—CH₂—. In an embodiment, Y is -(PEG)ₓ-, S1 is —CH₂—NH—CO—(CH₂)₄—, and S2 is —(CH₂)₀—CO—NH—CH₂—CH₂—.

In an embodiment, Y is -(PEG)ₓ-, S1 is —CH₂—NH—CO—(CH₂)₀—, and S2 is —(CH₂)₁—CO—NH—CH₂—CH₂—. In an embodiment, Y is -(PEG)ₓ-, S1 is —CH₂—NH—CO—(CH₂)₁—, and S2 is —(CH₂)₁—CO—NH—CH₂—CH₂—. In an embodiment, Y is -(PEG)ₓ-, S1 is —CH₂—NH—CO—(CH₂)₂—, and S2 is —(CH₂)₁—CO—NH—CH₂—CH₂—. In an embodiment, Y is -(PEG)ₓ-, S1 is —CH₂—NH— CO—(CH₂)₃—, and S2 is —(CH₂)₁—CO—NH—CH₂—CH₂—. In an embodiment, Y is -(PEG)ₓ-, S1 is —CH₂—NH—CO—(CH₂)₄—, and S2 is —(CH₂)₁—CO—NH—CH₂—CH₂—.

In an embodiment, Y is -(PEG)ₓ-, S1 is —CH₂—NH—CO—(CH₂)₀—, and S2 is —(CH₂)₂—CO—NH—CH₂—CH₂—. In an embodiment, Y is -(PEG)ₓ-, S1 is —CH₂—NH—CO—(CH₂)₁—, and S2 is —(CH₂)₂—CO—NH—CH₂—CH₂—. In an embodiment, Y is -(PEG)ₓ-, S1 is —CH₂—NH—CO—(CH₂)₂—, and S2 is —(CH₂)₂—CO—NH—CH₂—CH₂—. In an embodiment, Y is -(PEG)ₓ-, S1 is —CH₂—NH— CO—(CH₂)₃—, and S2 is —(CH₂)₂—CO—NH—CH₂—CH₂—. In an embodiment, Y is -(PEG)ₓ-, S1 is —CH₂—NH—CO—(CH₂)₄—, and S2 is —(CH₂)₂—CO—NH—CH₂—CH₂—.

In an embodiment, Y is -(PEG)ₓ-, S1 is —CH₂—NH—CO—(CH₂)₀—, and S2 is —(CH₂)₃—CO—NH—CH₂—CH₂—. In an embodiment, Y is -(PEG)ₓ-, S1 is —CH₂—NH—CO—(CH₂)₁—, and S2 is —(CH₂)₃—CO—NH—CH₂—CH₂—. In an embodiment, Y is -(PEG)ₓ-, S1 is —CH₂—NH—CO—(CH₂)₂—, and S2 is —(CH₂)₃—CO—NH—CH₂—CH₂—. In an embodiment, Y is -(PEG)ₓ-, S1 is —CH₂—NH— CO—(CH₂)₃—, and S2 is —(CH₂)₃—CO—NH—CH₂—CH₂—. In an embodiment, Y is -(PEG)ₓ-, S1 is —CH₂—NH—CO—(CH₂)₄—, and S2 is —(CH₂)₃—CO—NH—CH₂—CH₂—.

In an embodiment, Y is -(PEG)ₓ-, S1 is —CH₂—NH—CO—(CH₂)₀—, and S2 is —(CH₂)₄—CO—NH—CH₂—CH₂—. In an embodiment, Y is -(PEG)ₓ-, S1 is —CH₂—NH—CO—(CH₂)₁—, and S2 is —(CH₂)₄—CO—NH—CH₂—CH₂—. In an embodiment, Y is -(PEG)ₓ-, S1 is —CH₂—NH—CO—(CH₂)₂—, and S2 is —(CH₂)₄—CO—NH—CH₂—CH₂—. In an embodiment, Y is -(PEG)ₓ-, S1 is —CH₂—NH— CO—(CH₂)₃—, and S2 is —(CH₂)₄—CO—NH—CH₂—CH₂—. In an embodiment, Y is -(PEG)ₓ-, S1 is —CH₂—NH—CO—(CH₂)₄—, and S2 is —(CH₂)₄—CO—NH—CH₂—CH₂—.

In an embodiment, Y is -(PEG)ₓ-, S1 is —CH₂—NH—CO—(CH₂)₀—, and S2 is —(CH₂)₀—NH—CO—CH₂—CH₂—. In an embodiment, Y is -(PEG)ₓ-, S1 is —CH₂—NH—CO—(CH₂)₁—, and S2 is —(CH₂)₀—NH—CO—CH₂—CH₂—. In an embodiment, Y is -(PEG)ₓ-, S1 is —CH₂—NH—CO—(CH₂)₂—, and S2 is —(CH₂)₀—NH—CO—CH₂—CH₂—. In an embodiment, Y is -(PEG)ₓ-, S1 is —CH₂—NH— CO—(CH₂)₃—, and S2 is —(CH₂)₀—NH—CO—CH₂—CH₂—. In an embodiment, Y is -(PEG)ₓ-, S1 is —CH₂—NH—CO—(CH₂)₄—, and S2 is —(CH₂)₀—NH—CO—CH₂—CH₂—.

In an embodiment, Y is -(PEG)ₓ-, S1 is —CH₂—NH—CO—(CH₂)₀—, and S2 is —(CH₂)₁—NH—CO—CH₂—CH₂—. In an embodiment, Y is -(PEG)ₓ-, S1 is —CH₂—NH—CO—(CH₂)₁—, and S2 is —(CH₂)₁—NH—CO—CH₂—CH₂—. In an embodiment, Y is -(PEG)ₓ-, S1 is —CH₂—NH—CO—(CH₂)₂—, and S2 is —(CH₂)₁—NH—CO—CH₂—CH₂—. In an embodiment, Y is -(PEG)ₓ-, S1 is —CH₂—NH— CO—(CH₂)₃—, and S2 is —(CH₂)₁—NH—CO—CH₂—CH₂—. In an embodiment, Y is -(PEG)ₓ-, S1 is —CH₂—NH—CO—(CH₂)₄—, and S2 is —(CH₂)₁—NH—CO—CH₂—CH₂—.

In an embodiment, Y is -(PEG)ₓ-, S1 is —CH₂—NH—CO—(CH₂)₀—, and S2 is —(CH₂)₂—NH—CO—CH₂—CH₂—. In an embodiment, Y is -(PEG)ₓ-, S1 is —CH₂—NH—CO—(CH₂)₁—, and S2 is —(CH₂)₂—NH—CO—CH₂—CH₂—. In an embodiment, Y is -(PEG)ₓ-, S1 is —CH₂—NH—CO—(CH₂)₂—, and S2 is —(CH₂)₂—NH—CO—CH₂—CH₂—. In an embodiment, Y is -(PEG)ₓ-, S1 is —CH₂—NH— CO—(CH₂)₃—, and S2 is —(CH₂)₂—NH—CO—CH₂—CH₂—. In an embodiment, Y is -(PEG)ₓ-, S1 is —CH₂—NH—CO—(CH₂)₄—, and S2 is —(CH₂)₂—NH—CO—CH₂—CH₂—.

In an embodiment, Y is -(PEG)ₓ-, S1 is —CH₂—NH—CO—(CH₂)₀—, and S2 is —(CH₂)₃—NH—CO—CH₂—CH₂—. In an embodiment, Y is -(PEG)ₓ-, S1 is —CH₂—NH—CO—(CH₂)₁—, and S2 is —(CH₂)₃—NH—CO—CH₂—CH₂—. In an embodiment, Y is -(PEG)ₓ-, S1 is —CH₂—NH—CO—(CH₂)₂—, and S2 is —(CH₂)₃—NH—CO—CH₂—CH₂—. In an embodiment, Y is -(PEG)ₓ-, S1 is —CH₂—NH— CO—(CH₂)₃—, and S2 is —(CH₂)₃—NH—CO—CH₂—CH₂—. In an embodiment, Y is -(PEG)ₓ-, S1 is —CH₂—NH—CO—(CH₂)₄—, and S2 is —(CH₂)₃—NH—CO—CH₂—CH₂—.

In an embodiment, Y is -(PEG)ₓ-, S1 is —CH₂—NH—CO—(CH₂)₀—, and S2 is —(CH₂)₄—NH—CO—CH₂—CH₂—. In an embodiment, Y is -(PEG)ₓ-, S1 is —CH₂—NH—CO—(CH₂)₁—, and S2 is —(CH₂)₄—NH—CO—CH₂—CH₂—. In an embodiment, Y is -(PEG)ₓ-, S1 is —CH₂—NH—CO—(CH₂)₂—, and S2 is —(CH₂)₄—NH—CO—CH₂—CH₂—. In an embodiment, Y is -(PEG)ₓ-, S1 is —CH₂—NH— CO—(CH₂)₃—, and S2 is —(CH₂)₄—NH—CO—CH₂—CH₂—. In an embodiment, Y is -(PEG)ₓ-, S1 is —CH₂—NH—CO—(CH₂)₄—, and S2 is —(CH₂)₄—NH—CO—CH₂—CH₂—.

In an embodiment, Y is -(PEG)ₓ-, S1 is —CH₂—CO—NH—(CH₂)₀—, and S2 is —(CH₂)₀—CO—NH—CH₂—CH₂. In an embodiment, Y is -(PEG)ₓ-, S1 is —CH₂—CO—NH—(CH₂)₁—, and S2 is —(CH₂)₀—CO—NH—CH₂—CH₂. In an embodiment, Y is -(PEG)ₓ-, S1 is —CH₂—CO—NH—(CH₂)₂—, and S2 is —(CH₂)₀—CO—NH—CH₂—CH₂—. In an embodiment, Y is -(PEG)ₓ-, S1 is —CH₂—CO—NH—(CH₂)₃—, and S2 is —(CH₂)₀—CO—NH—CH₂—CH₂—. In an embodiment, Y is -(PEG)ₓ-, S1 is —CH₂—CO— NH—(CH₂)₄—, and S2 is —(CH₂)₀—CO—NH—CH₂—CH₂—.

In an embodiment, Y is -(PEG)ₓ-, S1 is —CH₂—CO—NH—(CH₂)₀—, and S2 is —(CH₂)₁—CO—NH—CH₂—CH₂. In an embodiment, Y is -(PEG)ₓ-, S1 is —CH₂—CO—NH—(CH₂)₁—, and S2 is —(CH₂)₁—CO—NH—CH₂—CH₂—. In an embodiment, Y is -(PEG)ₓ-, S1 is —CH₂—CO—NH—(CH₂)₂—, and S2 is —(CH₂)₁—CO—NH—CH₂—CH₂—. In an embodiment, Y is -(PEG)ₓ-, S1 is —CH₂—CO—NH—(CH₂)₃—, and S2 is —(CH₂)₁—CO—NH—CH₂—CH₂—. In an embodiment, Y is -(PEG)ₓ-, S1 is —CH₂—CO— NH—(CH₂)₄—, and S2 is —(CH₂)₁—CO—NH—CH₂—CH₂—.

In an embodiment, Y is -(PEG)ₓ-, S1 is —CH₂—CO—NH—(CH₂)₀—, and S2 is —(CH₂)₂—CO—NH—CH₂—CH₂—. In an embodiment, Y is -(PEG)ₓ-, S1 is —CH₂—CO—NH—(CH₂)₁—, and S2 is —(CH₂)₂—CO—NH—CH₂—CH₂—. In an embodiment, Y is -(PEG)ₓ-, S1 is —CH₂—CO—NH—(CH₂)₂—, and S2 is —(CH₂)₂—CO—NH—CH₂—CH₂—. In an embodiment, Y is -(PEG)ₓ-, S1 is —CH₂—CO— NH—(CH₂)₃—, and S2 is —(CH₂)₂—CO—NH—CH₂—CH₂—. In an embodiment, Y is -(PEG)ₓ-, S1 is —CH₂—CO—NH—(CH₂)₄—, and S2 is —(CH₂)₂—CO—NH—CH₂—CH₂—.

In an embodiment, Y is -(PEG)ₓ-, S1 is —CH₂—CO—NH—(CH₂)₀—, and S2 is —(CH₂)₃—CO—NH—CH₂—CH₂—. In an embodiment, Y is -(PEG)ₓ-, S1 is —CH₂—CO—NH—(CH₂)₁—, and S2 is —(CH₂)₃—CO—NH—CH₂—CH₂—. In an embodiment, Y is -(PEG)ₓ-, S1 is —CH₂—CO—NH—(CH₂)₂—, and S2 is —(CH₂)₃—CO—NH—CH₂—CH₂—. In an embodiment, Y is -(PEG)ₓ-, S1 is —CH₂—CO— NH—(CH₂)₃—, and S2 is —(CH₂)₃—CO—NH—CH₂—CH₂—. In an embodiment, Y is -(PEG)ₓ-, S1 is —CH₂—CO—NH—(CH₂)₄—, and S2 is —(CH₂)₃—CO—NH—CH₂—CH₂—.

In an embodiment, Y is -(PEG)ₓ-, S1 is —CH₂—CO—NH—(CH₂)₀—, and S2 is —(CH₂)₄—CO—NH—CH₂—CH₂—. In an embodiment, Y is -(PEG)ₓ-, S1 is —CH₂—CO—NH—(CH₂)₁—, and S2 is -(CH₂)₄—CO—NH—CH₂—CH₂—. In an embodiment, Y is -(PEG)ₓ-, S1 is —CH₂—CO—NH—(CH₂)₂—, and S2 is —(CH₂)₄—CO—NH—CH₂—CH₂—. In an embodiment, Y is -(PEG)ₓ-, S1 is —CH₂—CO— NH—(CH₂)₃—, and S2 is —(CH₂)₄—CO—NH—CH₂—CH₂—. In an embodiment, Y is -(PEG)ₓ-, S1 is —CH₂—CO—NH—(CH₂)₄—, and S2 is —(CH₂)₄—CO—NH—CH₂—CH₂—.

In an embodiment, Y is -(PEG)ₓ-, S1 is —CH₂—CO—NH—(CH₂)₀—, and S2 is —(CH₂)₀—NH—CO—CH₂—CH₂—. In an embodiment, Y is -(PEG)ₓ-, S1 is —CH₂—CO—NH—(CH₂)₁—, and S2 is —(CH₂)₀—NH—CO—CH₂—CH₂—. In an embodiment, Y is -(PEG)ₓ-, S1 is —CH₂—CO—NH—(CH₂)₂—, and S2 is —(CH₂)₀—NH—CO—CH₂—CH₂—. In an embodiment, Y is -(PEG)ₓ-, S1 is —CH₂—CO— NH—(CH₂)₃—, and S2 is —(CH₂)₀—NH—CO—CH₂—CH₂—. In an embodiment, Y is -(PEG)ₓ-, S1 is —CH₂—CO—NH—(CH₂)₄—, and S2 is —(CH₂)₀—NH—CO—CH₂—CH₂—.

In an embodiment, Y is -(PEG)ₓ-, S1 is —CH₂—CO—NH—(CH₂)₀—, and S2 is —(CH₂)₁—NH—CO—CH₂—CH₂—. In an embodiment, Y is -(PEG)ₓ-, S1 is —CH₂—CO—NH—(CH₂)₁—, and S2 is —(CH₂)₁—NH—CO—CH₂—CH₂—. In an embodiment, Y is -(PEG)ₓ-, S1 is —CH₂—CO—NH—(CH₂)₂—, and S2 is —(CH₂)₁—NH—CO—CH₂—CH₂—. In an embodiment, Y is -(PEG)ₓ-, S1 is —CH₂—CO— NH—(CH₂)₃—, and S2 is —(CH₂)₁—NH—CO—CH₂—CH₂—. In an embodiment, Y is -(PEG)ₓ-, S1 is —CH₂—CO—NH—(CH₂)₄—, and S2 is —(CH₂)₁—NH—CO—CH₂—CH₂—.

In an embodiment, Y is -(PEG)ₓ-, S1 is —CH₂—CO—NH—(CH₂)₀—, and S2 is —(CH₂)₂—NH—CO—CH₂—CH₂—. In an embodiment, Y is -(PEG)ₓ-, S1 is —CH₂—CO—NH—(CH₂)₁—, and S2 is —(CH₂)₂—NH—CO—CH₂—CH₂—. In an embodiment, Y is -(PEG)ₓ-, S1 is —CH₂—CO—NH—(CH₂)₂—, and S2 is —(CH₂)₂—NH—CO—CH₂—CH₂—. In an embodiment, Y is -(PEG)ₓ-, S1 is —CH₂—CO— NH—(CH₂)₃—, and S2 is —(CH₂)₂—NH—CO—CH₂—CH₂—. In an embodiment, Y is -(PEG)ₓ-, S1 is —CH₂—CO—NH—(CH₂)₄—, and S2 is —(CH₂)₂—NH—CO—CH₂—CH₂—.

In an embodiment, Y is -(PEG)ₓ-, S1 is —CH₂—CO—NH—(CH₂)₀—, and S2 is —(CH₂)₃—NH—CO—CH₂—CH₂—. In an embodiment, Y is -(PEG)ₓ-, S1 is —CH₂—CO—NH—(CH₂)₁—, and S2 is —(CH₂)₃—NH—CO—CH₂—CH₂—. In an embodiment, Y is -(PEG)ₓ-, S1 is —CH₂—CO—NH—(CH₂)₂—, and S2 is —(CH₂)₃—NH—CO—CH₂—CH₂—. In an embodiment, Y is -(PEG)ₓ-, S1 is —CH₂—CO— NH—(CH₂)₃—, and S2 is —(CH₂)₃—NH—CO—CH₂—CH₂—. In an embodiment, Y is -(PEG)ₓ-, S1 is —CH₂—CO—NH—(CH₂)₄—, and S2 is —(CH₂)₃—NH—CO—CH₂—CH₂—.

In an embodiment, Y is -(PEG)ₓ-, S1 is —CH₂—CO—NH—(CH₂)₀—, and S2 is —(CH₂)₄—NH—CO—CH₂—CH₂—. In an embodiment, Y is -(PEG)ₓ-, S1 is —CH₂—CO—NH—(CH₂)₁—, and S2 is —(CH₂)₄—NH—CO—CH₂—CH₂—. In an embodiment, Y is -(PEG)ₓ-, S1 is —CH₂—CO—NH—(CH₂)₂—, and S2 is —(CH₂)₄—NH—CO—CH₂—CH₂—. In an embodiment, Y is -(PEG)ₓ-, S1 is —CH₂—CO— NH—(CH₂)₃—, and S2 is —(CH₂)₄—NH—CO—CH₂—CH₂—. In an embodiment, Y is -(PEG)ₓ-, S1 is —CH₂—CO—NH—(CH₂)₄—, and S2 is —(CH₂)₄—NH—CO—CH₂—CH₂—.

In an embodiment, Y is a polyethylene glycol linker -(PEG)ₓ-, wherein (PEG)ₓ contains 4 repetitive units of polyethylene oxide —CH₂—CH₂—O— groups, S1 is —CH₂—NH—CO—(CH₂)_{z}—, and S2 is —(CH₂)_{f}—CO—NH—CH₂—CH₂—, wherein each z and f is independently 0 or 2.

In an embodiment, Y is a polyethylene glycol linker -(PEG)ₓ-, wherein (PEG)ₓ contains 4 repetitive units of polyethylene oxide —CH₂—CH₂—O— groups, S1 is —CH₂—NH—CO—(CH₂)_{z}—, and S2 is —(CH₂)_{f}—NH—CO—CH₂—CH₂—, wherein each z and f is independently 0 or 2.

In an embodiment, each R1 of the tracer compound is independently a methyl group, Y is a polyethylene glycol linker -(PEG)₄- containing 4 repetitive units of polyethylene oxide —CH₂—CH₂—O— groups, S1 is —CH₂—NH—CO—(CH₂)₀—, and S2 is —(CH₂)₂—CO—NH—CH₂—CH₂—, and R2 is H.

In an embodiment, each R1 of the tracer compound is independently a methyl group, Y is a polyethylene glycol linker -(PEG)₄- containing 4 repetitive units of polyethylene oxide —CH₂—CH₂—O— groups, S1 is —CH₂—NH—CO—(CH₂)₀—, and S2 is —(CH₂)₂—NH—CO—CH₂—CH₂—, and R2 is H.

In an embodiment, Y is -(PEG)₄-, S1 is —CH₂—NH—CO—(CH₂)₀—, and S2 is —(CH₂)₀—CO—NH—CH₂—CH₂—. In an embodiment, Y is -(PEG)₄-, S1 is —CH₂—NH—CO—(CH₂)₀—, and S2 is —(CH₂)₁—CO—NH—CH₂—CH₂—. In an embodiment, Y is -(PEG)₄-, S1 is —CH₂—NH—CO—(CH₂)₀—, and S2 is —(CH₂)₂—CO—NH—CH₂—CH₂—. In an embodiment, Y is -(PEG)₄-, S1 is —CH₂—NH—CO—(CH₂)₁—, and S2 is —(CH₂)₀—CO—NH—CH₂—CH₂—. In an embodiment, Y is -(PEG)₄-, S1 is —CH₂—NH— CO—(CH₂)₁—, and S2 is —(CH₂)₁—CO—NH—CH₂—CH₂—. In an embodiment, Y is -(PEG)₄-, S1 is —CH₂—NH—CO—(CH₂)₁—, and S2 is —(CH₂)₂—CO—NH—CH₂—CH₂—. In an embodiment, Y is -(PEG)₄-, S1 is —CH₂—NH—CO—(CH₂)₂—, and S2 is —(CH₂)₀—CO—NH—CH₂—CH₂—. In an embodiment, Y is -(PEG)₄-, S1 is —CH₂—NH—CO—(CH₂)₂—, and S2 is —(CH₂)₁—CO—NH—CH₂—CH₂—. In an embodiment, Y is -(PEG)₄-, S1 is —CH₂—NH—CO—(CH₂)₂—, and S2 is —(CH₂)₂—CO—NH—CH₂—CH₂—.

In an embodiment, Y is -(PEG)₄-, S1 is —CH₂—NH—CO—(CH₂)₀—, and S2 is —(CH₂)₀—NH—CO—CH₂—CH₂—. In an embodiment, Y is -(PEG)₄-, S1 is —CH₂—NH—CO—(CH₂)₀—, and S2 is —(CH₂)₁—NH—CO—CH₂—CH₂—. In an embodiment, Y is -(PEG)₄-, S1 is —CH₂—NH—CO—(CH₂)₀—, and S2 is —(CH₂)₂—NH—CO—CH₂—CH₂—. In an embodiment, Y is -(PEG)₄-, S1 is —CH₂—NH— CO—(CH₂)₁—, and S2 is —(CH₂)₀—NH—CO—CH₂—CH₂—. In an embodiment, Y is -(PEG)₄-, S1 is —CH₂—NH—CO—(CH₂)₁—, and S2 is —(CH₂)₁—NH—CO—CH₂—CH₂—. In an embodiment, Y is -(PEG)₄-, S1 is —CH₂—NH—CO—(CH₂)₁—, and S2 is —(CH₂)₂—NH—CO—CH₂—CH₂—. In an embodiment, Y is -(PEG)₄-, S1 is —CH₂—NH—CO—(CH₂)₂—, and S2 is —(CH₂)₀—NH—CO—CH₂—CH₂—. In an embodiment, Y is -(PEG)₄-, S1 is —CH₂—NH—CO—(CH₂)₂—, and S2 is —(CH₂)₁—NH—CO—CH₂—CH₂—. In an embodiment, Y is -(PEG)₄-, S1 is —CH₂—NH—CO—(CH₂)₂—, and S2 is —(CH₂)₂—NH—CO—CH₂—CH₂—.

In an embodiment, Y is -(PEG)₄-, S1 is —CH₂—CO—NH—(CH₂)₀—, and S2 is —(CH₂)₀—CO—NH—CH₂—CH₂—. In an embodiment, Y is -(PEG)₄-, S1 is —CH₂—CO—NH—(CH₂)₀—, and S2 is —(CH₂)₁—CO—NH—CH₂—CH₂—. In an embodiment, Y is -(PEG)₄-, S1 is —CH₂—CO—NH—(CH₂)₀—, and S2 is —(CH₂)₂—CO—NH—CH₂—CH₂—. In an embodiment, Y is -(PEG)₄-, S1 is —CH₂—CO— NH—(CH₂)₁—, and S2 is —(CH₂)₀—CO—NH—CH₂—CH₂—. In an embodiment, Y is -(PEG)₄-, S1 is —CH₂—CO—NH—(CH₂)₁—, and S2 is —(CH₂)₁—CO—NH—CH₂—CH₂—. In an embodiment, Y is -(PEG)₄-, S1 is —CH₂—CO—NH—(CH₂)₁—, and S2 is —(CH₂)₂—CO—NH—CH₂—CH₂—. In an embodiment, Y is -(PEG)₄-, S1 is —CH₂—CO—NH—(CH₂)₂—, and S2 is —(CH₂)₀—CO—NH—CH₂—CH₂—. In an embodiment, Y is -(PEG)₄-, S1 is —CH₂—CO—NH—(CH₂)₂—, and S2 is —(CH₂)₁—CO—NH—CH₂—CH₂—. In an embodiment, Y is -(PEG)₄-, S1 is —CH₂—CO—NH—(CH₂)₂—, and S2 is —(CH₂)₂—CO—NH—CH₂—CH₂—.

In an embodiment, Y is -(PEG)₄-, S1 is —CH₂—CO—NH—(CH₂)₀—, and S2 is —(CH₂)₀—NH—CO—CH₂—CH₂—. In an embodiment, Y is -(PEG)₄-, S1 is —CH₂—CO—NH—(CH₂)₀—, and S2 is —(CH₂)₁—NH—CO—CH₂—CH₂—. In an embodiment, Y is -(PEG)₄-, S1 is —CH₂—CO—NH—(CH₂)₀—, and S2 is —(CH₂)₂—NH—CO—CH₂—CH₂—. In an embodiment, Y is -(PEG)₄-, S1 is —CH₂—CO—NH—(CH₂)₁—, and S2 is —(CH₂)₀—NH—CO—CH₂—CH₂—. In an embodiment, Y is -(PEG)₄-, S1 is —CH₂—CO— NH—(CH₂)₁—, and S2 is —(CH₂)₁—NH—CO—CH₂—CH₂—. In an embodiment, Y is -(PEG)₄-, S1 is —CH₂—CO—NH—(CH₂)₁—, and S2 is —(CH₂)₂—NH—CO—CH₂—CH₂—. In an embodiment, Y is -(PEG)₄-, S1 is —CH₂—CO—NH—(CH₂)₂—, and S2 is —(CH₂)₀—NH—CO—CH₂—CH₂—. In an embodiment, Y is -(PEG)₄-, S1 is —CH₂—CO—NH—(CH₂)₂—, and S2 is —(CH₂)₁—NH—CO—CH₂—CH₂—. In an embodiment, Y is -(PEG)₄-, S1 is —CH₂—CO—NH—(CH₂)₂—, and S2 is —(CH₂)₂—NH—CO—CH₂—CH₂—.

In an embodiment, Y is a polyethylene glycol linker -(PEG)ₓ-, wherein (PEG)ₓ contains 9 repetitive units of polyethylene oxide —CH₂—CH₂—O— groups, S1 is —CH₂—NH—CO—(CH₂)_{z}—, and S2 is —(CH₂)_{f}—CO—NH—CH₂—CH₂—, wherein each z and f is independently 0 or 2.

In an embodiment, Y is a polyethylene glycol linker -(PEG)ₓ-, wherein (PEG)ₓ contains 9 repetitive units of polyethylene oxide —CH₂—CH₂—O— groups, S1 is —CH₂—NH—CO—(CH₂)_{z}—, and S2 is —(CH₂)_{f}—NH—CO—CH₂—CH₂—, wherein each z and f is independently 0 or 2.

In an embodiment, each R1 of the tracer compound is independently a methyl group, Y is a polyethylene glycol linker -(PEG)₉- containing 9 repetitive units of polyethylene oxide —CH₂—CH₂—O— groups, S1 is —CH₂—NH—CO—(CH₂)₀—, and S2 is —(CH₂)₂—CO—NO—CH₂—CH₂—, and R2 is H.

In an embodiment, each R1 of the tracer compound is independently a methyl group, Y is a polyethylene glycol linker -(PEG)₉- containing 9 repetitive units of polyethylene oxide —CH₂—CH₂—O— groups, S1 is —CH₂—NH—CO—(CH₂)₀—, and S2 is —(CH₂)₂—NH—CO—CH₂—CH₂—, and R2 is H.

In an embodiment, Y is -(PEG)₉-, S1 is —CH₂—NH—CO—(CH₂)₀—, and S2 is —(CH₂)₀—CO—NH—CH₂—CH₂—. In an embodiment, Y is -(PEG)₉-, S1 is —CH₂—NH—CO—(CH₂)₀—, and S2 is —(CH₂)₁—CO—NH—CH₂—CH₂—. In an embodiment, Y is -(PEG)₉-, S1 is —CH₂—NH—CO—(CH₂)₀—, and S2 is —(CH₂)₂—CO—NH—CH₂—CH₂—. In an embodiment, Y is -(PEG)₉-, S1 is —CH₂—NH— CO—(CH₂)₁—, and S2 is —(CH₂)₀—CO—NH—CH₂—CH₂—. In an embodiment, Y is -(PEG)₉-, S1 is —CH₂—NH—CO—(CH₂)₁—, and S2 is —(CH₂)₁—CO—NH—CH₂—CH₂—. In an embodiment, Y is -(PEG)₉-, S1 is —CH₂—NH—CO—(CH₂)₁—, and S2 is —(CH₂)₂—CO—NH—CH₂—CH₂—. In an embodiment, Y is -(PEG)₉-, S1 is —CH₂—NH—CO—(CH₂)₂—, and S2 is —(CH₂)₀—CO—NH—CH₂—CH₂—. In an embodiment, Y is -(PEG)₉-, S1 is —CH₂—NH—CO—(CH₂)₂—, and S2 is —(CH₂)₁—CO—NH—CH₂—CH₂—. In an embodiment, Y is -(PEG)₉-, S1 is —CH₂—NH—CO—(CH₂)₂—, and S2 is —(CH₂)₂—CO—NH—CH₂—CH₂—.

In an embodiment, Y is -(PEG)₉-, S1 is —CH₂—NH—CO—(CH₂)₀—, and S2 is —(CH₂)₀—NH—CO—CH₂—CH₂—. In an embodiment, Y is -(PEG)₉-, S1 is —CH₂—NH—CO—(CH₂)₀—, and S2 is —(CH₂)₁—NH—CO—CH₂—CH₂—. In an embodiment, Y is -(PEG)₉-, S1 is —CH₂—NH—CO—(CH₂)₀—, and S2 is —(CH₂)₂—NH—CO—CH₂—CH₂—. In an embodiment, Y is -(PEG)₉-, S1 is —CH₂—NH— CO—(CH₂)₁—, and S2 is —(CH₂)₀—NH—CO—CH₂—CH₂—. In an embodiment, Y is -(PEG) 9-, S1 is —CH₂—NH—CO—(CH₂)₁—, and S2 is —(CH₂)₁—NH—CO—CH₂—CH₂—. In an embodiment, Y is -(PEG)₉-, S1 is —CH₂—NH—CO—(CH₂)₁—, and S2 is —(CH₂)₂—NH—CO—CH₂—CH₂—. In an embodiment, Y is -(PEG)₉-, S1 is —CH₂—NH—CO—(CH₂)₂—, and S2 is —(CH₂)₀—NH—CO—CH₂—CH₂—. In an embodiment, Y is -(PEG)₉-, S1 is —CH₂—NH—CO—(CH₂)₂—, and S2 is —(CH₂)₁—NH—CO—CH₂—CH₂—. In an embodiment, Y is -(PEG)₉-, S1 is —CH₂—NH—CO—(CH₂)₂—, and S2 is —(CH₂)₂—NH—CO—CH₂—CH₂—.

In an embodiment, Y is -(PEG)₉-, S1 is —CH₂—CO—NH—(CH₂)₀—, and S2 is —(CH₂)₀—CO—NH—CH₂—CH₂—. In an embodiment, Y is -(PEG)₉-, S1 is —CH₂—CO—NH—(CH₂)₀—, and S2 is —(CH₂)₁—CO—NH—CH₂—CH₂—. In an embodiment, Y is -(PEG)₉-, S1 is —CH₂—CO—NH—(CH₂)₀—, and S2 is —(CH₂)₂—CO—NH—CH₂—CH₂—. In an embodiment, Y is -(PEG)₉-, S1 is —CH₂—CO— NH—(CH₂)₁—, and S2 is —(CH₂)₀—CO—NH—CH₂—CH₂—. In an embodiment, Y is -(PEG)₉-, S1 is —CH₂—CO—NH—(CH₂)₁—, and S2 is —(CH₂)₁—CO—NH—CH₂—CH₂—. In an embodiment, Y is -(PEG)₉-, S1 is —CH₂—CO—NH—(CH₂)₁—, and S2 is —(CH₂)₂—CO—NH—CH₂—CH₂—. In an embodiment, Y is -(PEG)₉-, S1 is —CH₂—CO—NH—(CH₂)₂—, and S2 is —(CH₂)₀—CO—NH—CH₂—CH₂—. In an embodiment, Y is -(PEG)₉-, S1 is —CH₂—CO—NH—(CH₂)₂—, and S2 is —(CH₂)₁—CO—NH—CH₂—CH₂—. In an embodiment, Y is -(PEG)₉-, S1 is —CH₂—CO—NH—(CH₂)₂—, and S2 is —(CH₂)₂—CO—NH—CH₂—CH₂—.

In an embodiment, Y is -(PEG)₉-, S1 is —CH₂—CO—NH—(CH₂)₀—, and S2 is —(CH₂)₀—NH—CO—CH₂—CH₂—. In an embodiment, Y is -(PEG)₉-, S1 is —CH₂—CO—NH—(CH₂)₀—, and S2 is —(CH₂)₁—NH—CO—CH₂—CH₂—. In an embodiment, Y is -(PEG)₉-, S1 is —CH₂—CO—NH—(CH₂)₀—, and S2 is —(CH₂)₂—NH—CO—CH₂—CH₂—. In an embodiment, Y is -(PEG)₉-, S1 is —CH₂—CO— NH—(CH₂)₁—, and S2 is —(CH₂)₀—NH—CO—CH₂—CH₂—. In an embodiment, Y is -(PEG)₉-, S1 is —CH₂—CO—NH—(CH₂)₁—, and S2 is —(CH₂)₁—NH—CO—CH₂—CH₂—. In an embodiment, Y is -(PEG)₉-, S1 is —CH₂—CO—NH—(CH₂)₁—, and S2 is —(CH₂)₂—NH—CO—CH₂—CH₂—. In an embodiment, Y is -(PEG)₉-, S1 is —CH₂—CO—NH—(CH₂)₂—, and S2 is —(CH₂)₀—NH—CO—CH₂—CH₂—. In an embodiment, Y is -(PEG)₉-, S1 is —CH₂—CO—NH—(CH₂)₂—, and S2 is —(CH₂)₁—NH—CO—CH₂—CH₂—. In an embodiment, Y is -(PEG)₉-, S1 is —CH₂—CO—NH—(CH₂)₂—, and S2 is —(CH₂)₂—NH—CO—CH₂—CH₂—.

In an embodiment, the R2 of the tetrazine moiety of the tracer compound is a phenyl substituent. In an embodiment, the R2 of the tetrazine moiety of the tracer compound is an alkyl substituent with a formula CₙH₂ₙ₊₁, wherein n is an integer selected from the range 0-2. In an embodiment, the R2 of the tetrazine moiety of the tracer compound is an alkyl substituent with a formula C₁H₃. In an embodiment, the R2 of the tetrazine moiety of the tracer compound is an alkyl substituent with a formula C₂H₅. In an embodiment, R2 of the tetrazine moiety of the tracer compound is hydrogen (H).

In an embodiment, the tetrazine moiety comprising the R2 substituent of the tracer compound is a 3-phenyl-1,2,4,5-tetrazine. In an embodiment, the tetrazine moiety comprising the R2 substituent of the tracer compound is a 3-phenyl-6-methyl-1,2,4,5-tetrazine. In an embodiment, the tetrazine moiety comprising the R2 substituent of the tracer compound is a 3-phenyl-6-ethyl-1,2,4,5-tetrazine. In an embodiment, the tetrazine moiety comprising the R2 substituent of the tracer compound is a 3-phenyl-6-phenyl-1,2,4,5-tetrazine.

In an embodiment, the substituent R2 is a non-interfering group with regard to reactivity of the IEDDA conjugation between the tracer compound and a TCO-derivatized targeting moiety. Non-interfering in this context means that the R2 does not fully or substantially prevent IEDDA conjugation.

In an embodiment, at least one F in the (BF3)⁻ moiety of the tracer compound is a ¹⁸F. In an embodiment, one F in the (BF3)⁻ moiety of the tracer compound is a ¹⁸F, whereas the remaining two are ¹⁹F.

In an embodiment, the adduct is obtained through inverse electron demand Diels-Alder reaction (IEDDA) of a trans-cyclooctene derivatized targeting moiety with a tetrazine moiety of a tracer compound.

In an embodiment, a tetrazine ring of the tetrazine moiety of the tracer compound is chemically bound to the TCO-moiety of the TCO-derivatized targeting moiety in the adduct.

In an embodiment, the targeting moiety of the adduct is a protein, a peptide, an antibody, an antibody fragment, or a nanoparticle. The targeting moiety of the adduct targets a specific biomolecule in vitro and in vivo, and conjugates with it through its sequence and/or 3D (surface) structure.

In an embodiment, at least one F in the (BF3)⁻ moiety of the adduct is a ¹⁸F. In an embodiment, one F in the (BF3)⁻ moiety of the adduct is a ¹⁸F, whereas the remaining two are ¹⁹F.

In an embodiment, the TCO moiety of the TCO-derivatized targeting moiety is directly linked to the targeting moiety. In certain other embodiments the TCO moiety of the TCO-derivatized targeting moiety is indirectly linked to the targeting moiety via a linking moiety such as a PEGₓ-chain or a polylysine chain. In an embodiment, the polylysine chain is a α-polylysine chain. In an embodiment, the polylysine chain is a ε-polylysine chain. In an embodiment, the polylysine chain is a poly-l-lysine chain.

In an embodiment, the adduct or a pharmaceutically acceptable salt or solvate thereof. has a structure according to formula (II):

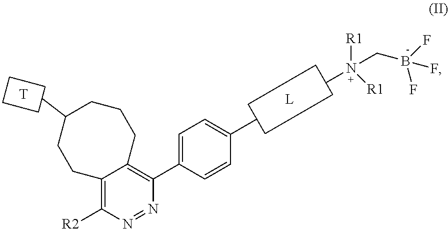

wherein each R1 is independently hydrogen (H) or an alkyl substituent with a formula CₙH₂ₙ₊₁, wherein n is an integer selected from the range 0-2; and
L is a linker moiety comprised of S1-Y—S2, wherein:
Y is (—CH₂—)ₘ wherein m is an integer selected from the range 1-4, or Y is a polyethylene glycol linker -(PEG)ₓ-, wherein (PEG)ₓ contains x repetitive units of polyethylene oxide —CH₂—CH₂—O— groups, and x is an integer selected from the range 1-20; and
S1 is —(CH₂)_{z}—CO—NH—(CH₂)_{z}, or S1 is —(CH₂)_{z}—NH—CO—(CH₂)_{z}—, wherein each z is independently an integer selected from the range 0-4; and
S2 is —CH₂—, or S2 is —(CH₂)_{f}—CO—NH—(CH₂)_{f}, or S2 is —(CH₂)_{f}—NH—CO—(CH₂)_{f}—, wherein each f is independently an integer selected from the range 0-4; and
R2 is either hydrogen (H) or a phenyl substituent or an alkyl substituent with a formula CₛH₂ₛ₊₁, wherein s is an integer selected from the range 0-2, and T comprises the targeting moiety of the adduct and optionally a linking moiety, wherein the linking moiety is a (PEG)ₓ-chain or a polylysine chain.

In an embodiment, the adduct is an IEDDA cycloaddition product of the TCO moiety of the TCO-derivatized targeting moiety and the tetrazine ring of the tracer compound and has the structure according to formula (11).

In an embodiment, in the formula (II) the groups R1, R2 and L have the same meaning as defined herein for the formula (I).

In an embodiment, the TCO-derivatized targeting moiety comprises a trans-cyclooctene moiety and the targeting moiety. In an embodiment, the TCO-derivatized targeting moiety comprises also other moieties, or side groups/chains besides the trans-cyclooctene moiety and the targeting moiety. In an embodiment, more than one linking moieties are positioned between the TCO moiety and the targeting moiety, thereby linking the TCO moiety and the targeting moiety together.

In an embodiment, a linking moiety between the TCO moiety and the targeting moiety is a polyethylene glycol linker containing repetitive units of polyethylene oxide —CH₂—CH₂—O— groups. In an embodiment, TCO-derivatized targeting moiety comprises a TCO-(PEG)ₓ-aldehyde, wherein x is an integer selected from the range 0-10. In an embodiment, TCO-derivatized targeting moiety comprises a TCO-(PEG)₀-aldehyde. In an embodiment, TCO-derivatized targeting moiety comprises a TCO-(PEG)₃-aldehyde. In an embodiment, TCO-derivatized targeting moiety comprises a TCO-(PEG)₄-aldehyde. In an embodiment, TCO-derivatized targeting moiety comprises a TCO-(PEG)₇-aldehyde.

In an embodiment, TCO-derivatized targeting moiety comprises a TCO-(PEG)ₓ-targeting moiety, wherein x is an integer selected from the range 0-10. In an embodiment, TCO-derivatized targeting moiety comprises a TCO-(PEG)₀-targeting moiety. In an embodiment, TCO-derivatized targeting moiety comprises a TCO-(PEG)₄-targeting moiety. In an embodiment, TCO-derivatized targeting moiety comprises a TCO-(PEG)₇-targeting moiety.

In an embodiment, the TCO-derivatized targeting moiety comprises a TCO-moiety and a Tyr³-octreotide (TOC) as the targeting moiety. In an embodiment, TCO-derivatized targeting moiety comprises a TCO-(PEG)ₓ-TOC, wherein x is an integer selected from the range 0-10. In an embodiment, TCO-derivatized targeting moiety comprises a TCO-(PEG)₄-TOC. In an embodiment, TCO-derivatized targeting moiety comprises a TCO-(PEG)₇-TOC. In an embodiment, the -(PEG)ₓ-linking moiety of the TCO-derivatized targeting moiety has the benefit of reducing the lipophilicity of the construct arising from the TCO moiety and the IEDDA cycloaddition product (Tz+TCO). In an embodiment, a polyethylene glycol linker -(PEG)ₓ- linking moiety between the TCO moiety and the targeting moiety is beneficial in modulating pharmacokinetics and metabolic stability of the final adduct. In an embodiment, a linking moiety between the TCO moiety and the targeting moiety is a polylysine linker, which is a biocompatible and biodegradable linker. In an embodiment, a polylysine linking moiety between the TCO moiety and the targeting moiety is also beneficial in modulating pharmacokinetics and metabolic stability of the final adduct.

In an embodiment, the structure and length of the linker moiety (L) of the tracer compound is optimized for ensuring optimal pharmacokinetics after conjugation of the radiolabeled adduct with a specific targeted entity. The optimal structure and length of the linker moiety of the tracer compound allows the adduct of the tracer compound and the TCO-derivatized targeting moiety, to obtain an optimal target-specific configuration once bound together, enabling a specific binding of the targeting moiety to the targeted entity. Accordingly, the modular structure and length of the linker moiety of the tracer compound, facilitates a high target-to-nontarget uptake ratio of the adduct in cells and tissues in vitro and in vivo. In an embodiment, the modular structure and length of the linker moiety of the tracer compound enables PET-imaging with good signal-to-noise ratios.

In an embodiment, the clearance of the tracer compound or the adduct occurs principally via the kidneys. In an embodiment, the linker moiety or the adduct is directed mostly to renal excretion in vivo.

In an embodiment, a method for manufacturing the tracer compound is disclosed, wherein the linker moiety of the starting material is comprised of S1-Y—S2, wherein:
Y is (—CH₂—)ₘ wherein m is an integer selected from the range 1-4, S1 is —CH₂—CO— NH—, or —CH₂—NH—CO— and S2 is —CH₂—; or
Y is a polyethylene glycol linker -(PEG)ₓ-, wherein (PEG)ₓ contains x repetitive units of polyethylene oxide —CH₂—CH₂—O— groups, and x is an integer selected from the range 1-20; and
S1 is —CH₂—NH—CO—(CH₂)_{z}—, or —CH₂—CO—NH—(CH₂)_{z}—, wherein each z is independently an integer selected from the range 0-4; and
S2 is —(CH₂)_{f}—CO—NH—CH₂—CH₂— or —(CH₂)_{f}—NH—CO—CH₂—CH₂—, wherein each f is independently an integer selected from the range 0-4.

In an embodiment, the IEDDA reaction speed depends on the IEDDA reaction partners conjugating together, and on the reaction conditions, the reaction conditions being determined at least by the concentration of the reagents, the reaction temperature, and the reaction pH. In an embodiment, the IEDDA conjugation of a tetrazine moiety of a tracer compound with a TCO moiety of the TCO-derivatized targeting moiety takes 30 min or less, preferably 20 min or less, more preferably 15 min or less, even more preferably 10 min or less, even more preferably 5 min or less. In a most preferred embodiment, the IEDDA conjugation of a tetrazine moiety of a tracer compound with a TCO moiety of the TCO-derivatized targeting moiety takes less than 1 min, preferably less than 30 sec, more preferably less than 15 sec, as the IEDDA reaction between tetrazine ring and TCO moiety is highly likely to occur within seconds. Nevertheless, in an embodiment the IEDDA conjugation of a tetrazine moiety of a tracer compound with a TCO moiety of the TCO-derivatized targeting moiety is carried out during 20-30 min at a temperature >+20° C., for improving the tautomeric homogeneity and stability of the resulting adduct.

In an embodiment, the IEDDA conjugation of a tetrazine moiety of a tracer compound with a TCO moiety of the TCO-derivatized targeting moiety, is carried out at ambient temperature (room temperature). In an embodiment, the IEDDA conjugation of a tetrazine moiety of a tracer compound with a TCO moiety of the TCO-derivatized targeting moiety is carried out efficiently at any temperature between +20° C. and +80° C.

In an embodiment, the method for manufacturing the adduct comprises allowing the tetrazine moiety of the radiolabeled tracer compound to react with the TCO moiety of the TCO-derivatized targeting moiety at a temperature between 20-80° C., preferably at a temperature between 40-70° C., more preferably at a temperature between 55-65° C., most preferably at a temperature 60° C. In an embodiment, the maximum reaction temperature for the IEDDA conjugation of a tetrazine moiety of a tracer compound with a TCO moiety of the TCO-derivatized targeting moiety is 80° C. In an embodiment, wherein the pretargeted PET imaging is utilized for imaging the adduct, the reaction temperature of the IEDDA conjugation is lower, the temperature being determined by the (body) temperature of the subject.

In an embodiment, the tautomeric homogeneity of the adduct is improved and amount of intermediate tautomers is reduced when the adduct is heated at a temperature >+20° C. In an embodiment, the tautomeric homogeneity of the adduct is improved when the adduct is heated at 20-80° C., preferably at 40-70° C., more preferably at 55-65° C., most preferably at 60° C. In an embodiment, the heating of the adduct at the said temperature is done for at least 5 min, preferably at least 10 min, or at least 15 min. In an embodiment, heating of the adduct increases the tautomeric homogeneity of the adduct and reduces the amount of intermediate tautomers of the adduct irreversibly. In an embodiment, the heating of the adduct prevents conversion of the adduct back to its intermediate tautomers after the heating and during storage at ambient room temperature of +20° C. In an embodiment, the said treatment with heating is utilized simultaneously with the IEDDA conjugation of the tetrazine moiety of the tracer compound with the TCO moiety of the TCO-derivatized targeting moiety. In an embodiment, the said treatment with heating is utilized after the IEDDA conjugation of the tetrazine moiety of the tracer compound with the TCO moiety of the TCO-derivatized targeting moiety.

In an embodiment, the solvent in the IEDDA conjugation of the tracer compound with the TCO-derivatized targeting moiety is water, an aqueous medium, an aqueous buffer solution, or a mixture of organic and aqueous solution, wherein the percentage of organic solvents is below 50%, preferably below 10%, and wherein the organic solvents comprise, for example, DMSO, ethanol, acetonitrile (MeCN), or methanol.

In an embodiment, the tetrazine moiety of the tracer compound reacts and IEDDA conjugates with the TCO moiety of the TCO-derivatized targeting moiety in acidic conditions to obtain the adduct. In an embodiment the tetrazine moiety of the tracer compound conjugates with the TCO moiety of the TCO-derivatized targeting moiety in conditions, wherein the pH is 2-7, preferably 2-4, most preferably 2-3.

In an embodiment, the radiolabeling reaction temperature of the tracer compound with ¹⁸F radioisotope is between 80-100° C., preferably between 80-90° C., more preferably between 85-90° C. In an embodiment, the radiolabeling reaction temperature of the tracer compound with ¹⁸F radioisotope is 85° C.

In an embodiment, the radiolabeling of the tracer compound with ¹⁸F radioisotope is carried out at a pH between 2.0-3.0.

In an embodiment, the radiolabeling reaction of the tracer compound with ¹⁸F radioisotope is carried out in acidic, pH-controlled buffer comprising a sufficient % of an organic solvent to dissolve the tracer, the organic solvent being, for example, MeCN or DMF. In an embodiment, the radiolabeling reaction of the tracer compound with at least one ¹⁸F radioisotope is carried out in pyridazine HCl buffer comprising an organic solvent, such as MeCN or DMF.

In an embodiment, the radiolabeling of the adduct with ¹⁸F radioisotope is carried out at a temperature between 80-100° C., preferably between 80-90° C., more preferably between 85-90° C. In an embodiment, the radiolabeling of the adduct with ¹⁸F radioisotope is carried out at a temperature of 85° C.

In an embodiment, the radiolabeling of the adduct with ¹⁸F radioisotope is carried out at a pH between 2.0-3.0.

In an embodiment, the radiolabeling reaction of the adduct with ¹⁸F radioisotope is carried out in acidic, pH-controlled buffer comprising a sufficient % of an organic solvent to dissolve the tracer, the organic solvent being, for example, MeCN or DMF. In an embodiment, the radiolabeling reaction of the adduct with ¹⁸F radioisotope is carried out in pyridazine HCl buffer comprising an organic solvent, such as MeCN or DMF.

In an embodiment, the adduct is radiolabeled after the IEDDA conjugation of a tracer compound with a TCO-derivatized targeting moiety. Accordingly, the replacement of the fluoride F with ¹⁸F radioisotope is carried out first after the IEDDA conjugation of the tracer compound to the TCO-derivatized targeting moiety. Having the entire adduct synthesized prior to radiolabeling allows radiolabeling the adduct shortly prior to use, thereby minimizing the decay of the radiolabel. In certain embodiments, by-products are created during IEDDA conjugation, which are laborious or impossible to remove from the reaction mix. Accordingly, radiolabeling the adduct after the IEDDA conjugation is beneficial for ensuring chemical purity of the resulting adduct.

In another embodiment, the zwitterion moiety of the tracer compound comprises a ¹⁸F radioisotope. Accordingly, at least one of the three fluorides (F) attached to the boron (B) of the zwitterion of the tracer compound is a ¹⁸F radioisotope. In an embodiment, the replacement of the fluoride F with ¹⁸F radioisotope is carried out prior to the IEDDA conjugation of the tracer compound to the TCO-derivatized targeting moiety. Certain targeting moieties are sensitive to the conditions required in ¹⁸F-radiolabeling. In such embodiments, the ¹⁸F radiolabeling of the tracer compound is carried out prior to IEDDA conjugation with the TCO-derivatized targeting moiety. Accordingly, in an embodiment wherein an adduct comprises a sensitive and/or fragile targeting moiety, the ¹⁸F radiolabeling of the tracer molecule is carried out prior to IEDDA conjugation. In an embodiment, such sensitive and/or fragile targeting moiety is an antibody or an enzyme. This is beneficial for ensuring integrity of the targeting moiety in the resulting adduct, as the said process order allows survival of the fragile targeting moieties from the radiolabeling process conditions.

In an embodiment, radiolabeling of the tracer compound prior to the IEDDA conjugation is utilized when the IEDDA conjugation between the tracer compound and the TCO-derivatized targeting moiety takes place inside a subject in vivo or in vitro. In this case the tracer compound is first brought in contact with the TCO-derivatized targeting moiety in the subject, after the TCO-derivatized targeting moiety has reached its target site. The radioimaging of the radiolabeled tracer compound employing this methodology is pretargeted PET imaging.

In an embodiment, is provided a use of the tracer compound and/or the adduct, in detection of targeted entities in a subject by radioimaging the subject, wherein the entities are targeted with the radiolabeled tracer compounds and/or the adduct. In an embodiment, is provided a method of detecting targeted entities in a subject by radioimaging the subject, wherein the entities are targeted with the radiolabeled tracer compound and/or the adduct.

In an embodiment, the tracer compound and/or the adduct is used in in vivo in radioimaging through systemic administration of the adduct into the subject. In an embodiment, the tracer compound and/or the adduct is used for detection of targeted entities in a subject by radioimaging, more specifically by positron emission tomography imaging, through imaging of radiolabeled targeted entities. In an embodiment, imaging of targeted entities refers to imaging of the radiolabeled tracer compounds and/or adducts which are capable of binding to selected targeted entities in vitro and in vivo.

In an embodiment, the tracer compound and/or the adduct is administered in an imaging-effective amount with regard to positron emission of the tracer compound and/or the adduct into a subject to be subjected to radioimaging.

In an embodiment, the tracer compound and/or the adduct is used in in vitro radioimaging of tissues and/or cells. In an embodiment, the tracer compound and/or the adduct is used for labelling of targeted entities in vitro and in vivo.

In an embodiment, the targeting moiety of the adduct can bind to a targeted entity in vitro. In an embodiment, the targeting moiety of the adduct can bind to a targeted entity in vivo. In an embodiment, the targeted entity is a biomolecule, such as a receptor, an enzyme, or a nanoparticle. In an embodiment, the targeted entity, to which the targeting moiety of the adduct can bind to, is an indicator of a specific physiological condition, such as cancer, neurodegeneration, inflammation, or infection

In an embodiment, an adduct comprising no targeting moiety or wherein the binding of a targeting moiety to a targeted entity has been blocked, has a low or nonsignificant binding to a targeted entity or a biomolecule in vitro. In an embodiment, an adduct comprising no targeting moiety or wherein the binding of a targeting moiety to a targeted entity has been blocked, has a low or nonsignificant binding to a targeted entity or a biomolecule in vivo.

In an embodiment, the tracer compound and the adduct have a good stability in plasma in vitro and in vivo. In an embodiment, the tracer compound and the adduct have a low bone uptake rate, indicating a good metabolic stability of the radiolabel of the tracer compound and the adduct. In an embodiment, the tracer compound and the adduct exhibit relatively low accumulation in non-targeted tissues.

In an embodiment, the radiolabeled tracer compound can be further processed and used in radioimaging up to 8 h, preferably up to 5 h, most preferably up to 2 h after radiolabeling the tracer compound. In an embodiment, the radiolabeled adduct which is an IEDDA cycloaddition product of the tracer compound and the TCO-derivatized targeting moiety, can be further processed and used in radioimaging up to 8 h, preferably up to 5 h, most preferably up to 2 h after radiolabeling the adduct.

In an embodiment, a kit for detection of targeted entities in a subject with radioimaging, comprises at least one compartment containing the tracer compound, at least one compartment containing at least one TCO-derivatized targeting moiety, and at least one compartment containing ¹⁸F for radiolabeling the tracer compound. In an embodiment, the kit comprises also aqueous and organic solvents for the IEDDA conjugation and radiolabeling of the tracer compound and/or the adduct. In an embodiment, the kit provides the necessary materials for radiolabeling the tracer compound prior to IEDDA conjugation of the adduct. In an embodiment, the kit provides the necessary materials for radiolabeling the adduct after the IEDDA conjugation of the tracer compound and the TCO-derivatized targeting moiety. In an embodiment, the kit is configured to be used in pretargeted PET imaging. In an embodiment, the kit provides all the necessary components for preparing the tracer compound and/or the adduct for detection of targeted entities in a subject. In an embodiment, the kit provides most of the necessary materials for preparing the tracer compound and/or the adduct for detection of targeted entities in a subject.

### EXAMPLES

Various embodiments have been presented. It should be appreciated that in this document, words comprise, include, and contain are each used as open-ended expressions with no intended exclusivity.

General

All reagents were purchased from commercial vendors and used as received without further purification. Tetrazines were purchased from Conju-Probe, BroadPharm or Jena Biosciences and iodoboronpinacol ester was purchased from Enamine. Sep-Pak C18-Light cartridges were purchased from Waters and PS—HCO₃-cartridges (Macherey-Nagel™ Chromafix™) from Fisher Scientific. No-carrier-added ¹⁸F-fluoride was produced in-house with an IBA 10/5 medical cyclotron from Hyox-18 ¹⁸O-enriched water purchased from Rotem Industries Limited (Arava, Israel). The synthesized precursors were analyzed with high-resolution mass spectrometry (HRMS) and nuclear magnetic resonance spectroscopy (NMR) analysis. The radiolabeled tracers were analyzed with radio-high-performance liquid chromatography (radio-HPLC). The excised organs were weighed and measured with a Wizard gamma counter. The 60 minutes dynamic positron emission tomography (PET) scans with computed tomography (CT) were acquired with a Molecubes PET (β-CUBE) coupled with a CT (γ-CUBE).

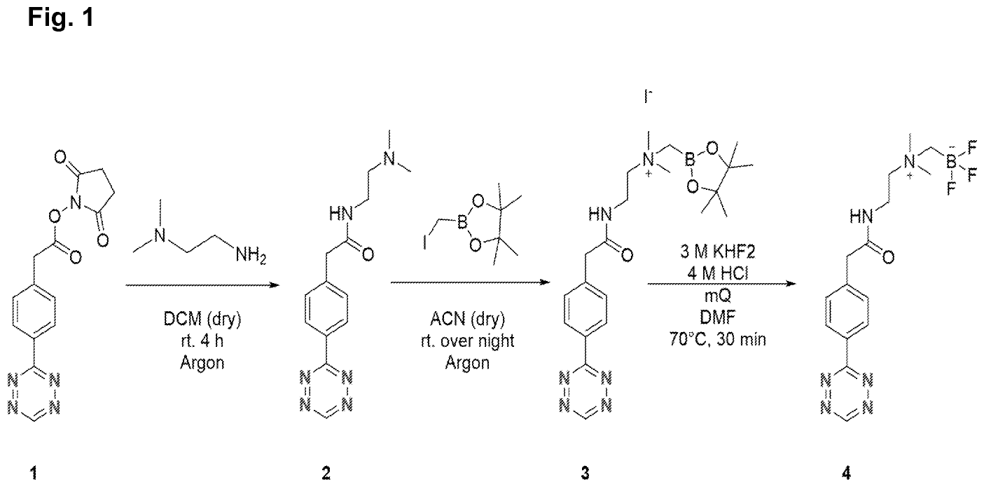

### Example 1. Synthesis of AmBF₃-Tz (Compound 4 of FIG. 1)

2-[4-(1,2,4,5-Tetrazin-3-yl)phenyl]-N-[2-(dimethylamino)ethyl]acetamide (2). N,N-Dimethylethylenediamine (13 μL, 0.12 mmol) was dissolved in 2 mL DCM under argon, followed by addition tetrazine NHS-ester (1) (25 mg, 0.08 mmol) in 3 mL DCM which was added dropwise into the clear solution. After stirring the mixture in room temperature for 1.5 hours, the crude reaction mixture was evaporated to dryness, re-suspended with 1 mL of ultrapure water (Milli-Q) and purified with a SEP-Pak Silica (elution with MeOH:DCM 1:9) to give a pink solid. Yield 68±26% (n=3) (11.5 mg, 0.04 mmol). ¹H NMR (300 MHz, Acetonitrile-d₃) δ 10.26 (s, 1H), 8.50 (d, J=8.4 Hz, 2H), 7.56 (d, J=8.2 Hz, 2H), 3.60 (s, 2H), 3.26 (s, 2H), 2.40 (s, 2H), 2.21 (s, 6H).

2-(2-(4-(1,2,4,5-Tetrazin-3-yl)phenyl)acetamido)-N,N-dimethyl-N-((4,4,5,5-tetramethyl-1,3,2-dioxaborolan-2-yl)methyl)ethan-1-aminium (3). Compound (2) (11.5 mg, 0.04 mmol) was dissolved into 1 mL of dry acetonitrile under argon followed by 2-(iodomethyl)-4,4,5,5-tetramethyl-1,3,2-dioxaborolane (10.8 mg, 0.04 mmol) in 300 μL of dry acetonitrile. The reaction mixture was stirred overnight and evaporated to dryness. Yield 58±31% (n=3) (11.5 mg, 0.04 mmol). ¹H NMR (300 MHz, Acetonitrile-d₃) δ 10.28 (s, 1H), 8.52 (d, J=8.3 Hz, 2H), 7.58 (d, J=8.2 Hz, 2H), 3.68 (s, 2H), 3.58 (s, 2H), 3.48 (s, 2H), 3.13 (s, 6H), 2.14 (s, 2H), 1.28 (s, 12H).

{[(2-{2-[4-(1,2,4,5-tetrazin-3-yl)phenyl]acetamido}ethyl)dimethylammonio]methyl}trifluoroborate (4). Compound (3) (0,043 mmol, 18 mg) was dissolved in a 15 mL Falcon (LDPE) tube with 1153 μL of DMF, followed by the addition of 387 μL of milli-Q water, 577 μL of 4 M HCl and 577 μL of 3 M KHF₂. The Falcon tube was closed and the reaction mixture was heated for 30 minutes in 70° C. and the fluorination reaction was closely monitored by HPLC (PDA detector 534 nm, 0.1% TFA-ACN:0.1% TFA-milli-Q water (80:20) isocratic 2.5 mL/min t_{R}(AmBF₃-Tz)=10.3 min) to avoid decomposition of the tetrazine. The reaction yielded a quantitative conversion of compound (3) to compound (4). The reaction mixture was diluted with 6 mL milli-Q water and added onto two parallel SPE C18 PLUS cartridge that were preconditioned with 5 mL ACN and 10 mL milli-Q water each. The C18 cartridges were washed with 20 mL on milli-Q water, dried with air and eluted with 1 mL of ACN to afford 13.9 mg of 4. ¹H NMR (400 MHz, CD₃CN) δ 10.30 (s, 1H), 8.54 (d, J=8.5 Hz, 2H), 7.58 (d, J=8.6 Hz, 2H), 3.68-3.56 (m, 4H), 3.34 (t, J=6.7 Hz, 2H), 3.01 (s, 6H), 2.38 (s, 2H). ¹¹B NMR (128 MHz, CD₃CN) δ 2.19, 1.80, 1.43, 1.03. ¹⁹F NMR (376 MHz, CD₃CN) δ −138.77, −138.89, −139.04, −139.17. ¹³C NMR (101 MHz, CD₃CN) δ 171.47, 167.25, 158.98, 141.95, 131.82, 131.42, 129.05, 118.30, 65.43, 54.32, 43.42, 34.75, 1.32. HRMS calculated for C₁₅H₂₁BF₃N₆O⁺ [M+H]⁺ 369.18165 m/z, found C₁₅H₂₁BF₃N₆O⁺ [M+H]⁺ 369.18134 m/z (mass error −0.85 ppm).

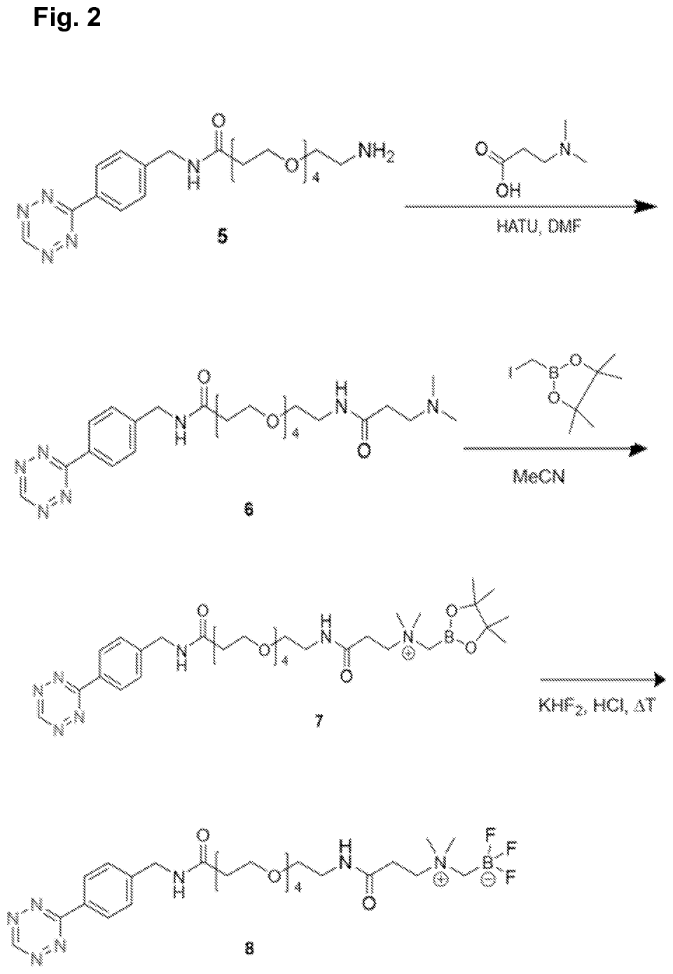

### Example 2. Synthesis of AmBF₃—PEG₄-Tz (Compound 8 of FIG. 2)

N-(4-(1,2,4,5-tetrazin-3-yl)benzyl)-1-(3-(dimethylamino)propanamido)-3,6,9,12-tetraoxapentadecan-15-amide (6). To 3-(dimethylamino) propanoic acid (4.8 mg, 31 μmol) in 0.3 mL DMF under argon atmosphere, HATU (8.5 mg, 23 μmol) in 0.1 mL DMF was added and stirred for 10 min at room temperature. N-(4-(1,2,4,5-tetrazin-3-yl)benzyl)-1-amino-3,6,9,12-tetraoxapentadecan-15-amide (10 mg, 21 μmol) (5) and DIPEA (30 μL) were added and the reaction was stirred for 2 h at room temperature. Solvents were evaporated and analysis by LC-MS showed a purity of >95%. LC-MS (+) calculated 534 m/z [M+H]+ for C₂₅H₄₀N₇O₅, found m/z (%)=534 (100) [M+H]+ t_{R}=8.5 min.

1-(4-(1,2,4,5-tetrazin-3-yl)phenyl)-N,N-dimethyl-3,19-dioxo-N-((4,4,5,5-tetramethyl-1,3,2-dioxaborolan-2-yl)methyl)-6,9,12,15-tetraoxa-2,18-diazahenicosan-21-aminium (7). Compound (6) (2 mg, 3.56 μmol) was dissolved into 200 μL of dry acetonitrile under argon followed by 2-(iodomethyl)-4,4,5,5-tetramethyl-1,3,2-dioxaborolane (1.01 mg, 3.7 μmol) in 100 μL of dry acetonitrile. The reaction mixture was stirred for 20 min and evaporated to dryness.

24-(4-(1,2,4,5-tetrazin-3-yl)phenyl)-1,1,1-trifluoro-3,3-dimethyl-6,22-dioxo-10,13,16,19-tetraoxa-3,7,23-triaza-1-boratetracosan-3-ium-1-uide (8). Without further purification, compound (7) (3.56 μmol) was dissolved in a 15 mL Falcon (LDPE) tube with 14.94 μL of DMF, followed by the addition of 4.93 μL of milli-Q water, 7.47 μL of 4 M HCl and 7.47 μL of 3 M KHF₂. The Falcon tube was closed, and the reaction mixture was heated for 10 minutes in 85° C. The reaction mixture was diluted with 6 mL milli-Q water and added onto two parallel SPE C18 PLUS cartridges that were preconditioned with 5 mL ACN and 10 mL milli-Q each. The C18 cartridges were washed with 20 mL on milli-Q, dried with air and eluted with 1 mL of ACN to afford 1.2 mg (1.95 μmol) of compound (8). Solvents were evaporated and analysis by LC-MS showed a purity of >95%. LC-MS (+) calculated 596 m/z [M−F]⁺ for C₂₆H₄₁BF₂N₇O₆, found m/z (%)=596 (100) [M−F]+, t_{R}=11.5 min. 1H NMR (400 MHz, Acetone-d6) δ 10.43 (s, 1H), 8.54 (s, 2H), 7.63 (s, 2H), 5.35 (s, 2H), 4.58 (s, 2H), 3.78 (s, 3H), 3.60 (s, 16H), 3.35 (s, 2H), 3.10 (s, 6H), 2.51 (s, 3H), 2.33 (s, 3H), 2.21 (s, 2H). 19F NMR (376 MHz, Acetonitrile-d3) δ −138.98, −139.12, −139.25.

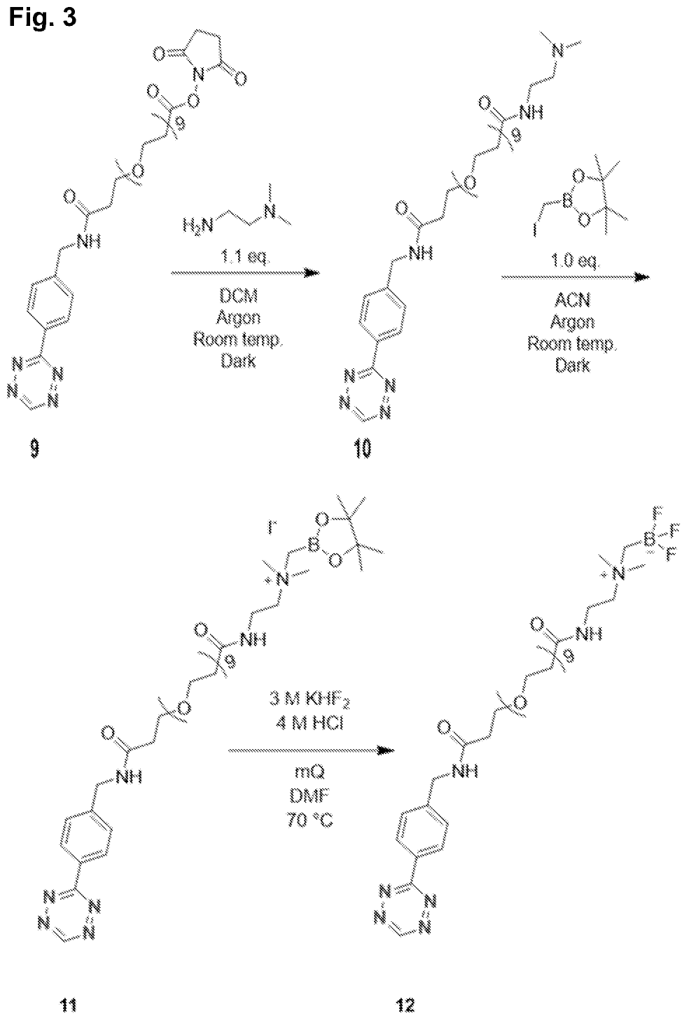

### Example 3. Synthesis of AmBF₃-PEG₉-Tz (compound 12 of FIG. 3)

N¹-(4-(1,2,4,5-tetrazin-3-yl)benzyl)-N³¹-(2-(dimethylamino)ethyl)-4,7,10,13,16,19,22,25,28-nonaoxahentriacontanediamide (10). Dimethylethylenediamine (0.677 mg, 7.7 μmol) was dissolved in 400 μL DCM under argon, followed by addition tetrazine-PEGg-NHS-ester (9) (5 mg, 6.4 μmol) in 600 μL DCM which was added dropwise into the clear solution. The reaction was monitored with TLC (RP-TLC, ACN:milli-Q water (80:20), R_{f}=tetrazine 0.83, R_{f}=Amine 0.00, R_{f}=tetrazine-amine 0.28). After stirring the mixture in room temperature for 20 minutes, the crude reaction mixture was loaded onto 3×C18 cartridges, dried with air and eluted into 4 fractions with 3 mL ACN. Pure fractions were combined and evaporated to dryness to give a pink solid. Yield >98% (1.3 mg, 0.0016 mmol). ¹H NMR (400 MHz, Acetonitrile-d₃) δ 10.28 (s, 1H), 8.53 (d, J=8.5 Hz, 2H), 7.57 (d, J=8.7 Hz, 2H), 5.36 (s, 1H), 4.50 (d, J=6.2 Hz, 2H), 3.74 (t, J=6.0 Hz, 2H), 3.67 (t, J=6.0 Hz, 2H), 3.59 (d, J=0.9 Hz, 30H), 3.47 (q, J=5.7 Hz, 2H), 3.29 (s, 1H), 3.04 (s, 2H), 2.73 (s, 6H), 2.48 (t, J=6.0 Hz, 3H), 2.39 (t, J=6.0 Hz, 3H).

1-(4-(1,2,4,5-tetrazin-3-yl)phenyl)-N,N-dimethyl-3,33-dioxo-N-((4,4,5,5-tetramethyl-1,3,2-dioxaborolan-2-yl)methyl)-6,9,12,15,18,21,24,27,30-nonaoxa-2,34-diazahexatriacontan-36-aminium (11). 2-(iodomethyl)-4,4,5,5-tetramethyl-1,3,2-dioxaborolane (0.0017 mmol, 0.46 mg) was dissolved in dry acetonitrile and added dropwise into a stirred solution of compound (10) (0.0017 mmol, 1.3 mg) in ACN under argon atmosphere overnight. The reaction was monitored with HPLC (PDA-detector 534 nm). The reaction mixture was evaporated to dryness and used as such for the subsequent fluorination reaction.

39-(4-(1,2,4,5-tetrazin-3-yl)phenyl)-1,1,1-trifluoro-3,3-dimethyl-7,37-dioxo-10,13,16,19,22,25,28,31,34-nonaoxa-3,6,38-triaza-1-boranonatriacontan-3-ium-1-uide (12). Compound (11) (0.0017 mmol, 1.74 mg) was dissolved in a 15 mL Falcon (LDPE) tube with 45.6 μL of DMF, followed by the addition of 15.5 μL of milli-Q water, 22.8 μL of 4 M HCl and 22.8 μL of 3 M KHF₂. The Falcon tube was closed and the reaction mixture was heated for 30 minutes in 70° C. and the fluorination reaction was closely monitored by HPLC (PDA detector, 534 nm) to avoid decomposition of the tetrazine. The reaction yielded a complete conversion of compound (11) to compound (12). The reaction mixture was diluted with 1 mL milli-Q water and added onto a SPE C18 Light cartridge (preconditioning: 5 mL ACN and 10 mL milli-Q water). The C18 cartridge was washed with 10 mL of milli-Q, dried with air and eluted with 200 μL of ACN to afford 1.9 mg of compound (12). ¹H NMR (400 MHz, CD₃CN) δ 10.29 (s, 1H), 8.56-8.54 (d, 2H), 7.60-7.58 (d, 2H), 7.22 (s, broad, 1H), 6.88 (s, broad, 1H), 4.53-4.51 (d, 2H), 3.75 (t, 2H), 3.67 (t, 2H), 3.62-3.56 (m, 32H), 3.33 (t, 2H), 3.03 (s, 6H), 2.49 (t, 2H), 2.38 (t, 2H). ¹⁹F NMR (376 MHz, CD₃CN) δ −138.80, −138.97, −139.08. ¹³C-NMR (101 MHz, CD₃CN). HRMS Calculated for C₃₆H₆₂BF₃N₇O₁₁⁺ [M+H]⁺ 836.45470 m/z, found C₃₆H₆₂BF₃N₇O₁₁⁺ [M+H]⁺ 836.45538 m/z (mass error 0.82 ppm).

### Example 4. Synthesis of Trans-Cyclooctene Aldehyde (TCO—CHO) (Compound 15 of

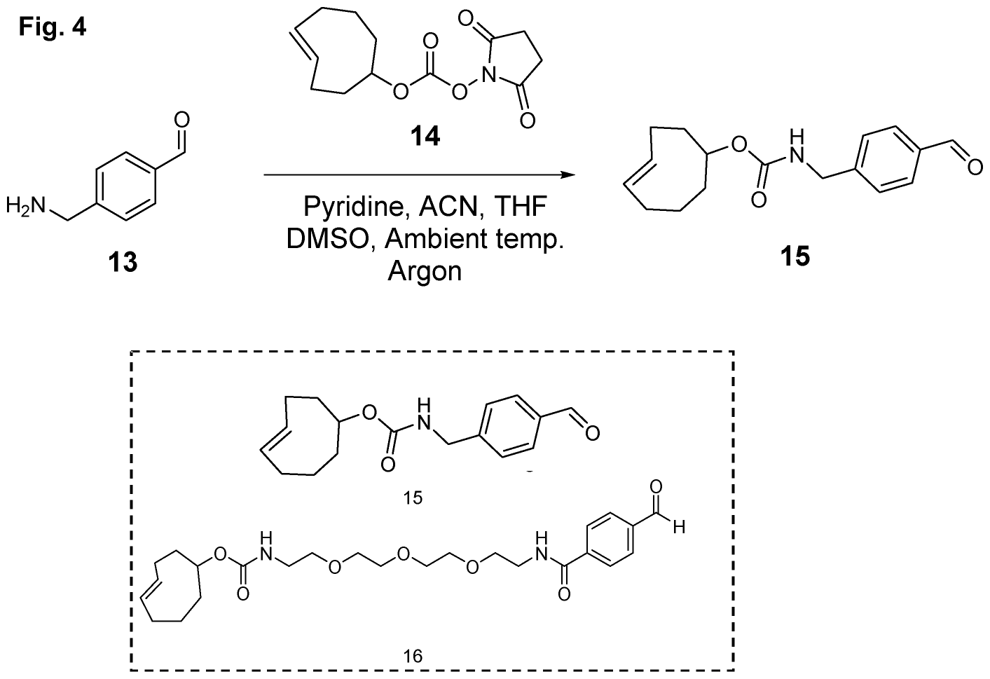

FIG. 4). 15.6 mg (91 nmol, 1.5 eq) of compound (13) as presented in the FIG. 4, was dissolved in 500 μL of THF and 150 μL DMSO under argon, followed by addition of 9.7 mg pyridine (122 nmol, 2.0 eq.) in 100 μL of THF and mixing of the solution for to 10 min. 16.3 mg (61 nmol, 1.0 eq.) of compound (14) was added dropwise and the solution was stirred overnight in room temperature. The reaction was monitored with normal phase TLC with ethylacetate:cyclohexane (1:1) as mobile phase and stained with KMnO₄-staining solution (t_{R} (pyridine)=0.00, t_{R} (1)=0.00, t_{R} (2)=0.90 RT (3)=0.80). The crude mixture was purified with a Sep-Pak SPE-Sil cartridge (preconditioned with 50 mL of ultrapure water) in-order to remove pyridine and unreacted compound (14). The mixture was pushed through SPE-Sil cartridge (fraction 1) and eluted with 1 mL of DCM (fraction 2). The collected fractions were further purified by semi-preparative HPLC (Phenomenex Alltima C18 column, 80% ACN+0.1% TFA isocratic 3 mL/min) where compound (15) eluted at t_{R}=6 min. LC-MS (+) m/z (%)=288.36 (27) [M+H]⁺, 310.30 (19) [M+Na]⁺ t_{R}=9.3 min, ¹H NMR (400 MHz, CDCl₃) 5 ppm, 10.00, 7.86, 7.84, 7.45, 7.43, 5.53, 4.99, 4.41, 2.35, 1.97, 1.75, 1.57, 1.27, 1.26. ¹³C NMR (101 MHz, CDCl₃) δ ppm, 191.81, 145.79, 135.64, 134.89, 133.01, 130.13, 127.77, 81.14, 44.70, 41.14, 38.67, 34.27, 32.50, 30.96.

### Example 5. General Procedure for Functionalizing Aminooxy-Functionalized Peptides (α-MSH-ONH₂, Exendin-4-ONH₂, Tyr³-Octreotide-ONH₂) with Trans-Cyclooctene

The aminooxy-functionalized custom-synthesized peptide (1 eq.) was dissolved into 600 μL of 0.3 M anilinium acetate buffer (pH 4.6). Commercially available trans-cyclooctene-PEG₃-aldehyde (FIG. 4, compound 16) (1.5 eq.) was dissolved in 17 μL chloroform and added dropwise into the stirred peptide solution. The reaction was monitored with HPLC (PDA-detector 280 nm). The functionalized peptide was purified with HPLC (MeCN(B)—H₂O(A)⁺0.1% TFA; 20-30-20% B, 30 min). t_{R}=α-MSH-TCO; 23 min, t_{R} TOC-TCO; 25 min, t_{R}=Exendin-4-TCO; 15.5 min. The MeCN in the fractions collected from HPLC was evaporated with pressurized air and the fractions containing mainly water were frozen (−80° C.). The frozen fractions were lyophilized dry and used as such afterwards. The fractions, when needed, were used as such and mixed with the selected tetrazine immediately after collecting from HPLC. In such an occasion the solution was diluted so that it contained >95% water during IEDDA cycloaddition.

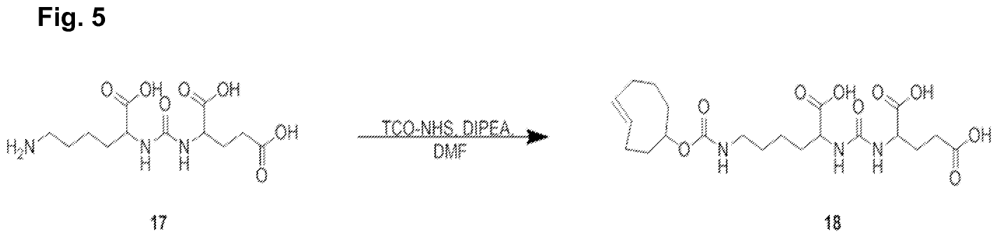

### Example 6. Synthesis of PSMA-Trans-Cyclooctene (Compound 18, FIG. 5)

To TCO—NHS (4.5 mg, 17 μmol) in dry DMF (300 μL) and DIPEA (3.2 mg, 25 μmol) under argon atmosphere, PSMA-amine (17) (5 mg, 15.7 μmol) in dry DMF (250 μL) was added dropwise and stirred overnight. PSMA-TCO (18) was purified by HPLC yielding 5.3 mg (71%). LC-MS (+) m/z (%)=472.5 (100) [M+H]⁺, 320.3 (96) [M−TCO-formate]+t_{R}=10.3 min. 1H NMR (400 MHz, CD₃OD) δ (ppm)=5.59 (m, 1H), 5.50 (m, 1H), 4.31 (m, 1H), 4.25 (s, 1H), 3.31 (s, 2H), 3.06 (m, 2H), 2.41 (m, 2H), 2.33 (m, 2H), 2.33 (m, 2H), 2.15 (m, 2H), 1.94 (m, 6H), 1.68 (m, 4H), 1.40 (m, 4H), 1.29 (m, 2H), 13C NMR (101 MHz, CD₃OD) δ (ppm)=136.10, 133.76, 81.59, 54.07, 53.59, 42.23, 39.67, 35.18, 33.49, 33.25, 32.11, 31.15, 30.55, 29.03, 23.86.

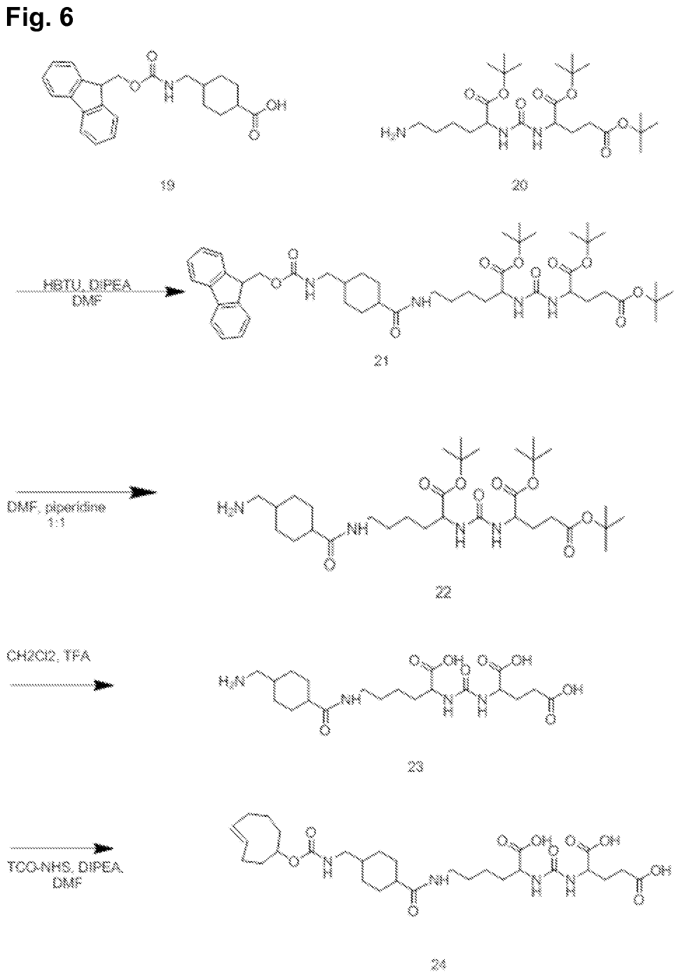

### Example 7. Synthesis of PSMA-Tranexamic Acid-Trans-Cyclooctene (Compound 24, FIG. 6)

di-tert-butyl ((6-(4-(((((9H-fluoren-9-yl)methoxy)carbonyl)amino)methyl)cyclohexane-1-carboxamido)-1-(tert-butoxy)-1-oxohexan-2-yl)carbamoyl)glutamate (21). To HBTU (76.6 mg, 201.5 μmol) and DIPEA (26.4 mg, 206 μmol) in dry DMF (400 μl), Fmoc-tranexamic acid (19) (78 mg, 206 μmol) was added in dry DMF (600 μL) and stirred for 10 min under argon atmosphere. Di-tert-butyl ((6-amino-1-(tert-butoxy)-1-oxohexan-2-yl)carbamoyl)glutamate (20) (25 mg, 51.5 μmol) was added in dry DMF (400 μL) and stirred under argon for 2 h. LC-MS (+) m/z (%)=850.1 (4) [M+H]⁺, 872.1 (3) [M+Na]+t_{R}=18.5 min.

di-tert-butyl ((6-(4-(aminomethyl)cyclohexane-1-carboxamido)-1-(tert-butoxy)-1-oxohexan-2-yl)carbamoyl)glutamate (22). Without further purification, 1.4 mL piperidine were added to (21) and stirred for >10 min at room temp. Solvents were evaporated and product was extracted with 5 mL Ethyl acetate and 3×2 mL brine solution. LC-MS (+) m/z (%)=628.0 (100) [M+H]⁺ t_{R}.=11.3 min.

((5-(4-(aminomethyl)cyclohexane-1-carboxamido)-1-carboxypentyl)carbamoyl)glutamic acid (23). Without further purification (22) was dissolved in 3 mL CH₂Cl₂/TFA (1:1) and stirred for 90 min. at room temp. Product was purified by HPLC (t_{R}=7.2 min) yielding 10.2 mg (43%). LC-MS (+) m/z (%)=459 (100) [M+H]+t_{R}.=2.6 min.

(E)-((1-carboxy-5-(4-((((cyclooct-4-en-1-yloxy)carbonyl)amino)methyl)cyclohexane-1-carboxamido)pentyl)carbamoyl)glutamic acid (24). To TCO—NHS (13 mg, 49 μmol) in dry DMF (600 μL) and DIPEA (8.9 mg, 70 μmol) (23) was added dropwise in 250 μL dry DMF and stirred overnight under argon atmosphere. Product was purified by HPLC (t_{R}=4.5 min) yielding 5.63 mg (41%). LC-MS (+) m/z (%)=611 (100) [M+H]+t_{R}=11.4 min. 1H NMR (400 MHz, CD₃OD) δ (ppm)=5.61 (m, 1H), 5.52 (m, 1H), 4.32 (m, 2H), 4.26 (m, 1H), 3.17 (m, 2H), 3.01 (s, 1H), 2.92 (m, 2H), 2.88 (s, 1H), 2.43 (m, 2H), 2.34 (m, 2H), 2.15 (m, 2H), 1.98 (m, 4H), 1.80 (m, 5H), 1.70 (m, 4H), 1.51 (m, 2H), 1.44 (m, 5H), 0.98 (m, 2H), 13C NMR (101 MHz, CD₃OD) δ (ppm)=136.10, 133.77, 53.94, 53.50, 46.47, 42.24, 39.92, 39.65, 39.09, 35.18, 33.50, 33.19, 32.11, 30.95, 30.24, 29.97, 28.93, 26.45, 23.89.

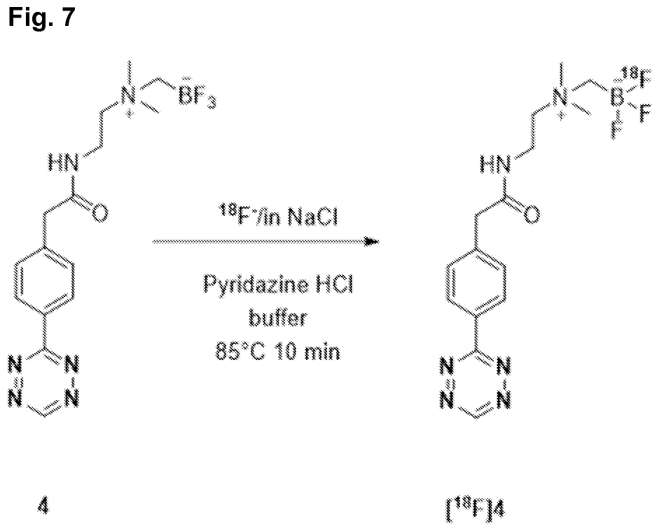

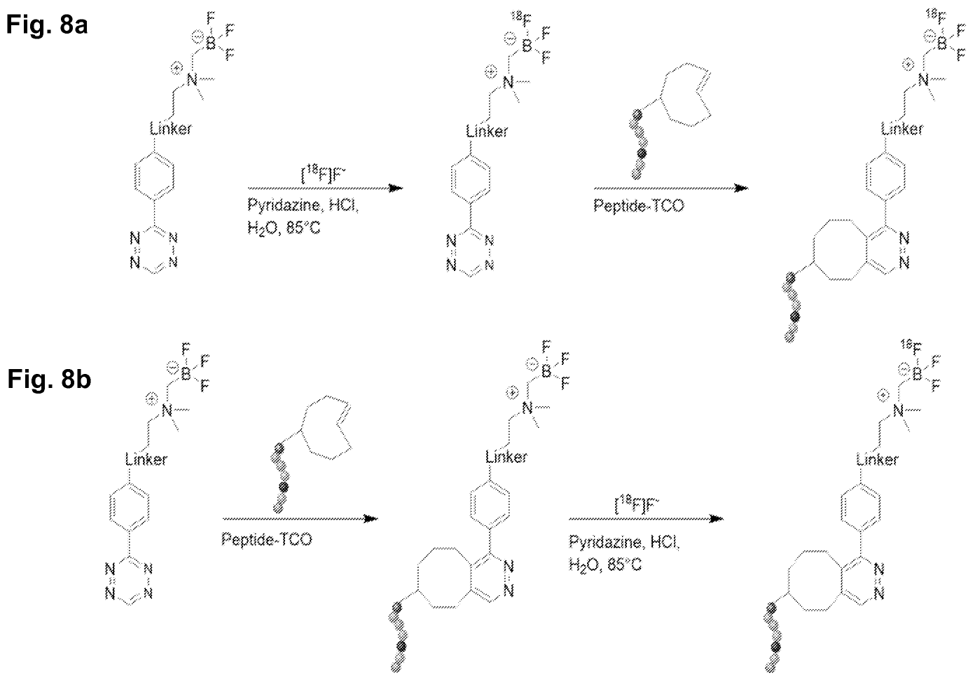

### Example 8. Procedure a) Radiolabeling of [¹⁸F]4 (AmBF₃-Tz), [¹⁸F]8 and [¹⁸F]12 Before IEDDA Conjugation

[¹⁸F]Fluoride was eluted into the reaction vial as ¹⁸F—NaF with 150 μL of 0.9% NaCl and concentrated at 125° C. under argon gas flow for 10 minutes to reach 10-25 μL reaction volume. Tetrazine (100 nmol) in 5 μL of acetonitrile was added into a polypropylene tube containing 10 μL of pyridazine HCl buffer (pH 2.0). The reaction mixture was heated for an additional 10 minutes at 83° C. and quenched with 600 uL of milliQ:EtOH (50:50). Alternatively, [¹⁸F]fluoride was trapped on a PS—HCO₃ cartridge and eluted (100 μL, pyridazine HCl buffer, pH 2.0) into the tube containing the tetrazine. The mixture was concentrated at 85° C. under argon flow (t=15 min) until it reached ˜10-20 μL, and quenched with ultrapure water (600 μL), and purified with a Sep-Pak C18 cartridge, providing the tracer. The procedure of radiolabeling the compound 4 (AmBF₃-Tz) with ¹⁸F radioisotope resulting in [¹⁸F]4 is presented in the FIG. 7. An example, of the procedure a) is shown in the FIG. 8a.

### Example 9. Procedure b) Pre-IEDDA Conjugation Before Radiolabeling of AmBF₃-Tz

To Tetrazine-AmBF₃ 4, 8 or 12 (1.85 μmol) in 20 μL dry acetonitrile was added the TCO-functionalized peptide 18 or 24 in equimolar amounts in milli-Q water (800 μL). The reaction was heated to 60° C. for 20 min. Product was purified by HPLC affording (28) (49%) (t_{R}=7.9 min) LC-MS (+) m/z (%)=811 (100) [M+H]+t_{R}=8.8 min or (29) (48%) (t_{R}=9.5 min) LC-MS (+) m/z (%)=950 (100) [M+H]+t_{R}=9.5 min. An example of the synthesis route according to the procedure b) is shown in the FIG. 8b.

### Example 10. IEDDA Conjugation of TCO Functionalized Peptides Tyr³-Octreotide (25), α-MSH, Exendin-4, PSMA (18) and PSMA-tranexamic acid (24), with [¹⁸F]AmBF₃-Tz ([¹⁸F]4) or its PEGylated derivatives [¹⁸F]8 or [¹⁸F]12, yielding the products [¹⁸F]AmBF₃-Tyr³-octreotide ([¹⁸F]25), [¹⁸F]AmBF₃-α-MSH ([¹⁸F]26), [¹⁸F]AmBF₃-Exendin-4 ([¹⁸F]27) and [¹⁸F]AmBF₃—PEG₉-Exendin-4 ([¹⁸F]30), [¹⁸F]AmBF₃—PSMA ([¹⁸F]28) and [¹⁸F]AmBF₃—PSMA-tranexamic acid ([¹⁸F]29)

The functionalization of the peptides (α-MSH—ONH₂, Exendin-4-ONH₂, Tyr³-octreotide-ONH₂) with trans-cyclooctene was done as described in the Example 5. Trans-cyclooctene functionalized peptide (20-50 μL in milli-Q water, 50 nmol) was added into the reaction mixture (20 μL) of a radiolabeled tetrazine and heated in 60° C. for 15 minutes. The reaction mixture was diluted with milli-Q water and purified with two C18 cartridges by washing with ultrapure milli-Q water (45 mL) and eluted with 150 μL of ethanol and 200 μL of 0.01 M PBS. The purified peptide solution was diluted to with 0.01 M PBS to contain 5% ethanol for intravenous administration. The crude mixtures were analyzed with HPLC: MeCN(B)—H₂O(A)⁺0.1% TFA 20-30-20% B for 30 min. The retention times on HPLC: t_{R} for compound [¹⁸F]26 ([¹⁸F]AmBF₃-α-MSH); 14.7 min, t_{R} for compound [¹⁸F]25 ([¹⁸F]AmBF₃-Tyr³-octreotide); 17.5 min, t_{R} for compound [¹⁸F]27 ([¹⁸F]AmBF₃-Exendin-4); 15.0-16.0 min, t_{R} for compound [¹⁸F]30 ([¹⁸F]AmBF₃—PEG₉-Exendin-4), 15.8-16.5 min. HRMS (E/Z)-[¹⁸F]25 found [M+H+Na]²⁺ 1048.49255 (−0.0855 ppm). These results demonstrate that variety of different peptides can be radiolabeled quickly in mild conditions by using the tracer compound.

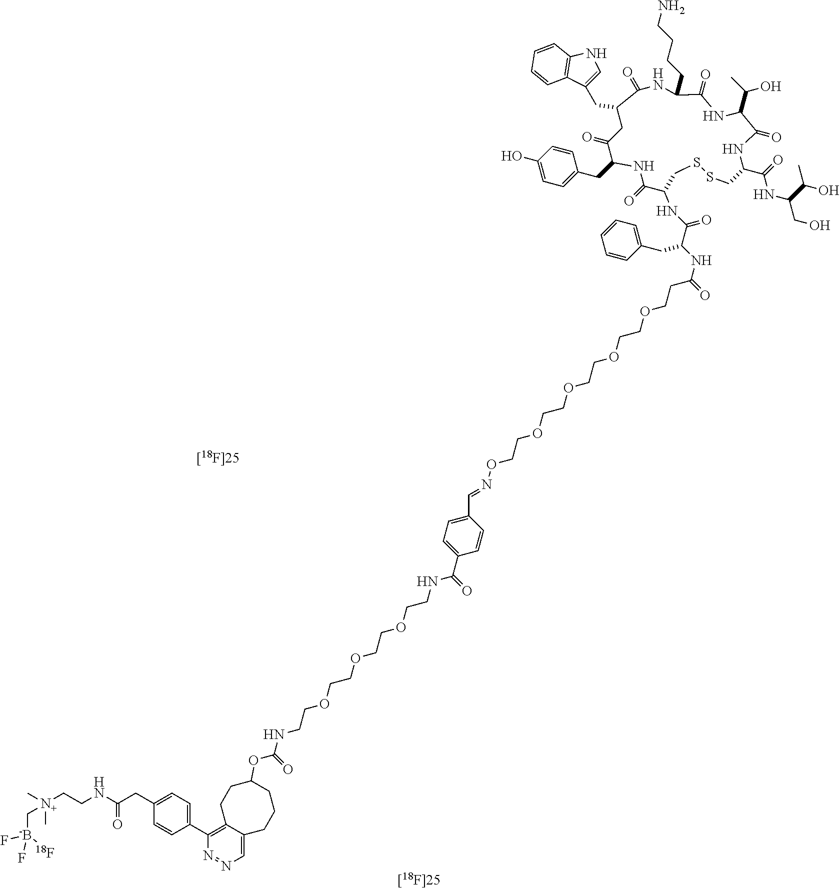

### Example 11. Radiolabeling of Pre-IEDDA Products

Fluorine-18 (1.8 GBq) was eluted from a PS—HCO₃ cartridge with 100 μL 0.9% NaCl solution or with pyridazine HCl buffer (pH 2, 100 uL) and evaporated to a volume of 10-15 μL at 100° C. (0.9% NaCl) or at 80-85° C. when using pyridazine HCl buffer. 28 or 29 (100 nmol) in 10 μL pyridazine buffer (pH=2) were added through an external line and the resulting solution was heated to 85° C. for 10 min. After dilution with 10 mL milli-Q water, activity was loaded on a preconditioned C18 cartridge and washed with additional 40 mL milli-Q water before elution with 400 μL 50% EtOH/PBS afforded [¹⁸F]28 (RCY: 5.2±1%) with a specific activity of (9.2±3.8 GBq/μmol) and [¹⁸F]29 (RCY: 11.8±3.1%) with a specific activity of (16.3±4.3 GBq/μmol).

### Example 12. Cells and Cell-Culturing

Rat pancreatic tumor cell line AR42J that expresses SSTR was obtained from American Type Culture Collection (Manassas, VA). C4-2 cells (ATCC® CRL-3314™) were cultured in DMEM medium (Gibco) supplemented with 18% F12 medium (Sigma), 10% FBS (Gibco) and 1% T-medium. Both cell lines were grown in at 37° C. in a humidified incubator containing 5% C02. Cells grown to 80%-90% confluence were used for either in vitro or in vivo experiments. Mouse skin melanoma B16/F10 cells were cultured in CO₂ Independent Medium (Life Technologies Gibco, cat. #18045054) supplemented with GlutaMax (1× final concentration, 10% FBS and Pen-Strep at 37° C. in a humidified incubator. The B16/F10 cell viability was 97%. The C4-2 cells (ATCC® CRL-3314™) were cultured in DMEM medium (Gibco) supplemented with 18% F12 medium (Sigma), 10% FBS (Gibco) and 1% T-medium. Both cell lines were grown in at 37° C. in a humidified incubator containing 5% C02. Cells grown to 80%-90% confluence were used for either in vitro or in vivo experiments.

### Example 13. Nonspecific B16/F10 Melanoma Cell-Uptake of [¹⁸F]AmBF₃-Tz ([¹⁸F]4)

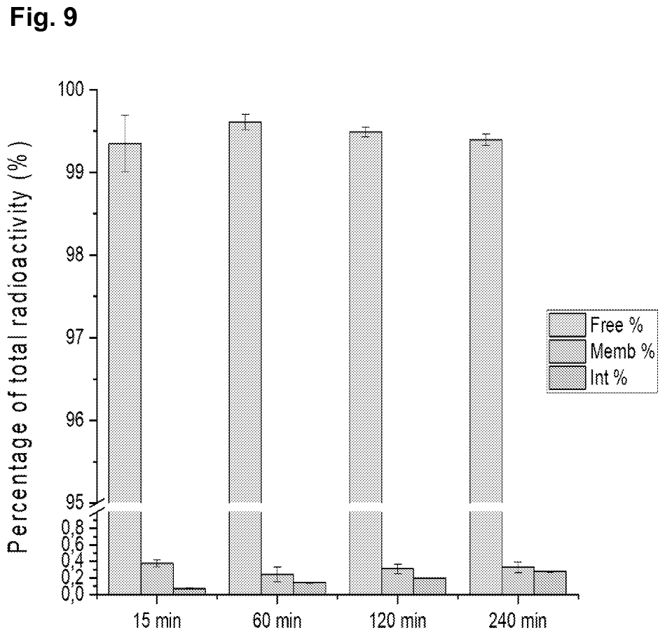

500 000 cells/well were seeded overnight on 6-well plates. The growth media was removed and the reaction media containing the radiotracer [¹⁸F]4 was added. For determining the amount of the radiotracer in the free fraction, at the designated time-points (15, 30, 60 and 120 minutes) the reaction media was removed and collected to a microtube, followed by washing the cells with 1 mL of cold 1×PBS and collecting the supernatant into the same microtube. The membrane-bound fraction was collected by adding cold glycine buffer (1 mL) on the cells, incubating for 5 minutes on an ice bath, removing the supernatant, repeating the procedure, and washing the cells with cold 1×PBS, and collecting all the supernatants to the same microtube. For determining the internalized fraction, 1 M NaOH was added on the cells and left to incubate in ambient temperature for 10 minutes. The supernatant was removed and the cells were washed twice with cold 1×PBS, and the supernatants were collected into the same microtube. The supernatants collected separately in each phase, were measure with a gamma counter for determining the radioactivity-ratio (%) of each fraction. Based on the determined radioactivity distribution between free, membrane-bound and internalized fractions, it was clearly shown that [¹⁸F]4 did not demonstrate nonspecific uptake in B16/F10 cells but stayed in the free fraction outside the cells throughout the study (from 99.3±0.09% at 15 min to 99.3±0.08% at 240 min, n=3). The nonspecific cell uptake of the adduct [¹⁸F]4 demonstrates that the tracer compound or the adduct, used to radiolabel a targeting moiety, does not bind to entities on the cell membrane or internalize to cells without a targeting moiety, such as a peptide. The radioactivity distribution between the aforementioned fractions is depicted in FIG. 9.

### Example 14. AR42J Cell-Uptake of TCO Functionalized Tyr³-Octreotide Conjugated with [¹⁸F]AmBF₃-Tz ([¹⁸F]AmBF₃-Tyr³-Octreotide, [¹⁸F]25)

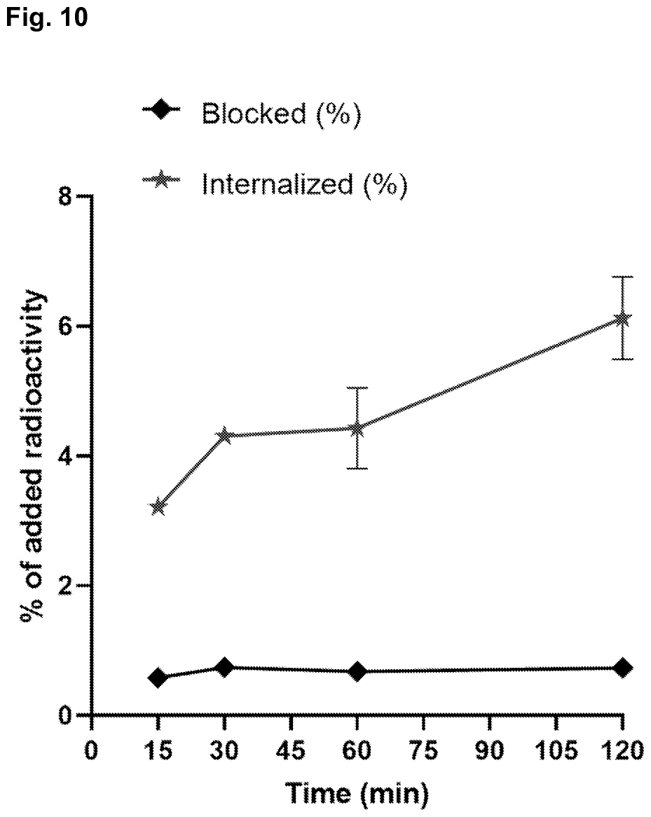

One million cells/well were seeded overnight on 6-well plates. The growth media was removed and the reaction media containing the radiotracer [¹⁸F]25 was added. In-order to study the specificity of the cell-uptake, a set of cells were co-incubated in the presence of 1 μM solution of non-modified octreotide. The non-modified octreotide used as a blocking octreotide, comprised only the octreotide peptide, and was not conjugated to TCO moiety or to a tracer compound, and thereby was not radiolabeled. For determining the amount of the radiotracer in the free fraction, at the designated time-points (15, 30, 60 and 120 minutes) the reaction media was removed and collected to a microtube, followed by washing the cells with 1 mL of cold 1×PBS and collecting the supernatant into the same microtube. The membrane-bound fraction was collected by adding cold glycine buffer (1 mL) on the cells, incubating for 5 minutes on ice, removing the supernatant, repeating the procedure and washing the cells with cold 1×PBS, and collecting all the supernatants to the same microtube. For determining the internalized fraction, 1 M NaOH was added on the cells and left to incubate in ambient temperature for 10 minutes. The supernatant was removed, cells were washed twice with cold 1×PBS, and the supernatants were collected into the same microtube. The supernatants collected separately in each phase, were measured with a gamma counter for determining the radioactivity % of each fraction. Based on the determined radioactivity distribution between free, membrane-bound and internalized fractions, it was clear that the cell uptake of [¹⁸F]25 was specific. The uptake (Internalized): from 3.21±0.06% at 15 min to 6.12±0.63% at 240 min, n=3) was efficiently blocked (blocked: from 0.58±0.11% at 15 min to 0.73±0.04% at 240 min, n=3) by an excess of non-modified octreotide. The blocking of the cell uptake of [¹⁸F]25 was efficient throughout the study, while the uptake in non-blocked conditions continued to grow as a function of time. The radioactivity distribution between the aforementioned fractions in non-blocked (internalized) and in blocked conditions are depicted in FIG. 10. These results with AR42J cells and compound [¹⁸F]25 demonstrate that the internalization of the radiolabeled adduct is target-specific (in this example to somatostatin receptors) and can be prevented by blocking the access of the targeting moiety of the adduct to the respective targeted entity.

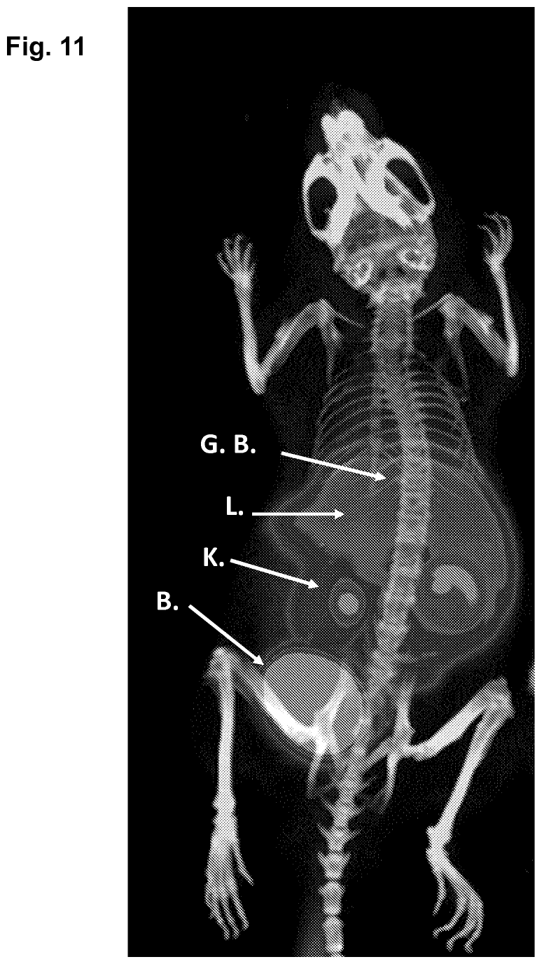

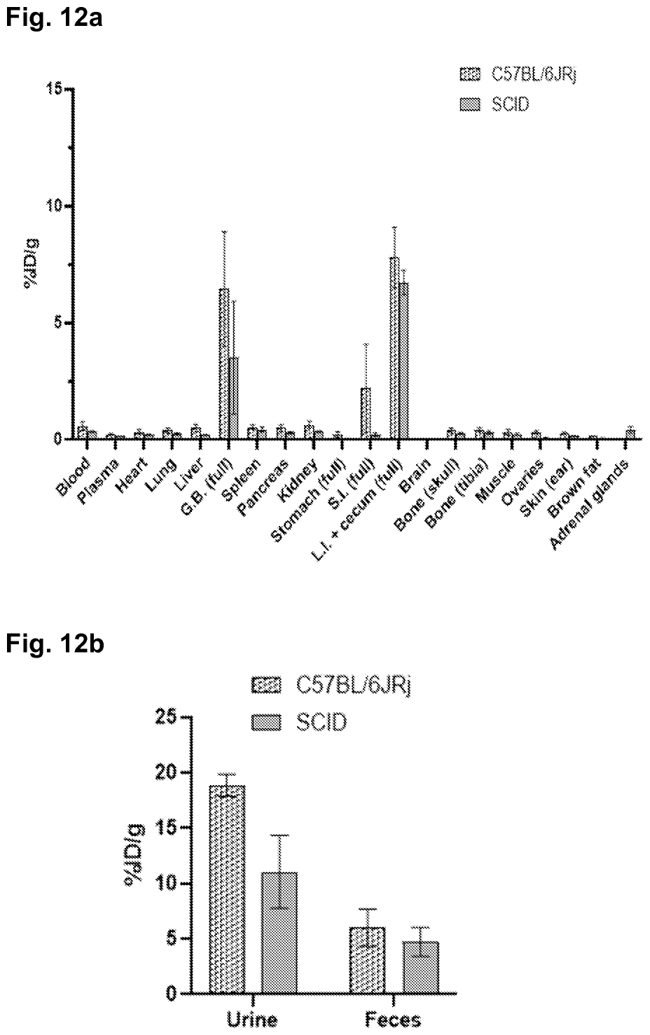

### Example 15. PET/CT Scan of [¹⁸F]AmBF₃-Tz ([¹⁸F]4) in Control SCID Mice

[¹⁸F]AmBF₃-Tz ([¹⁸F]4) was formulated in 10% ethanol in 0.01 M PBS and administered intravenously to SCID mice. The PET/CT image was acquired with Inveon PET/CT and Molecubes PET and CT. In the PEC/CT (FIG. 11) and biodistribution studies (FIGS. 12a and 12b) the tracer ([¹⁸F]4) demonstrated excellent stability proven by the lack of bone uptake. The main elimination route for the tracer was through kidneys, with minor accumulation in the liver and gallbladder (FIG. 11) illustrating the profile of an optimal prosthetic group.

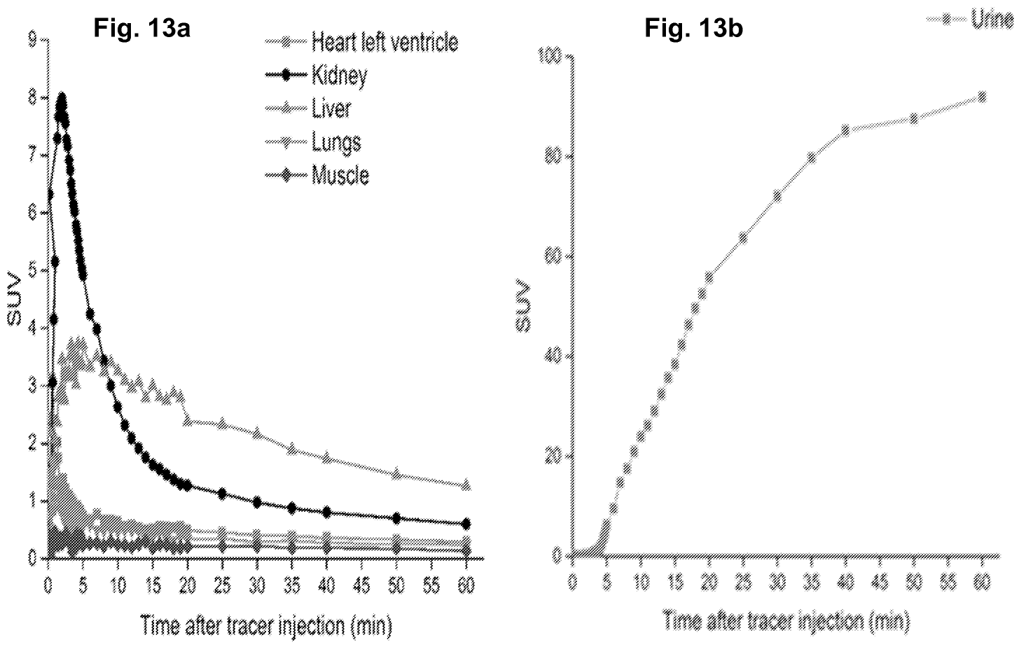

### Example 16. Elimination of Radioactivity after Intravenous Administration of [¹⁸F]AmBF₃-Tz ([¹⁸F]4) and Excretion of Radioactivity into Urine after Intravenous Administration of [¹⁸F]AmBF₃-Tz ([¹⁸F]4) in SCID Mice

[¹⁸F]AmBF₃-Tz ([¹⁸F]4) was formulated in 10% ethanol in 0.01 M PBS and administered intravenously to SCID mice. The standardized uptake values (SUVs) presenting the elimination profile (FIGS. 13a and 13b) were determined from the PET image by drawing regions-of-interest (ROI) around selected organs (heart, liver, kidney, lung, muscle, urinary bladder) and by measuring the ratio of radioactivity per unit volume of that ROI and normalized to the injected dose. The tracer ([¹⁸F]4) demonstrated excellent stability proven by the lack of bone uptake. The main elimination route for the tracer was through kidneys, with only minute accumulation in the liver and gallbladder illustrating the profile of an optimal prosthetic group. The elimination of radioactivity as a function of time in the mouse tissues (FIG. 13a. and 13b) indicates the adduct is eliminated quickly mainly via kidneys.

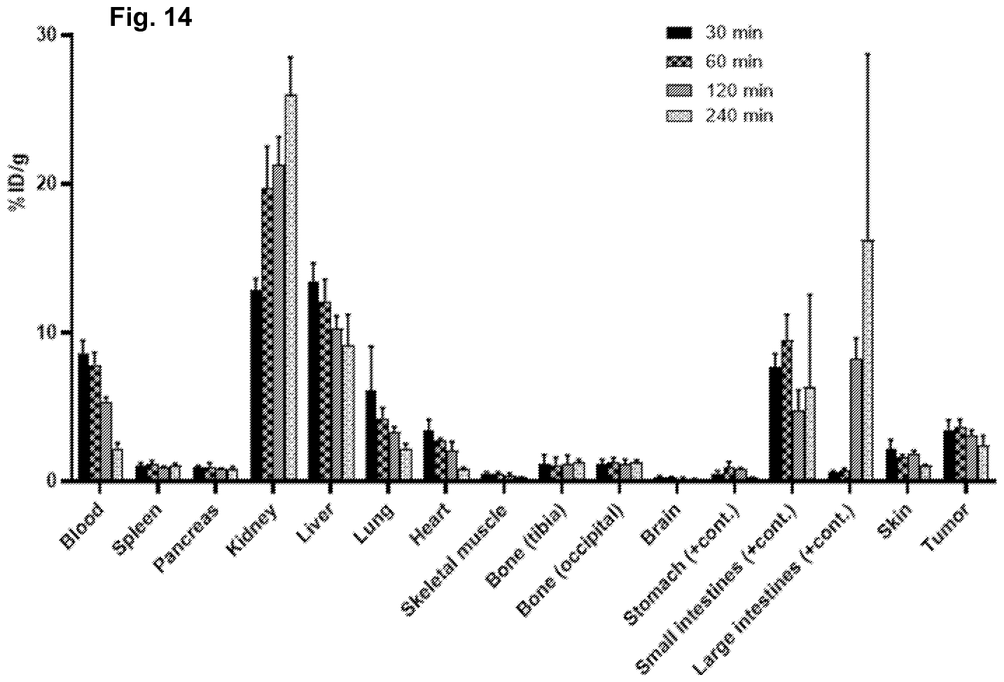

### Example 17. Biodistribution of [¹⁸F]25

[¹⁸F]AmBF₃-Tyr³-octreotide ([¹⁸F]25, 0.2 nmol, 150 μL, ˜1 MBq) was formulated in 4% ethanol in 0.01 M PBS and administered intravenously to AR42J tumor bearing Rj:NMRI-Foxn1 nu/nu mice. At predetermined time points after administration (t=30, 60, 120 and 240 min) selected organs were extracted, washed with water and blotted dry before subjecting to gammacounting. Based on the gammacounting data, the percentage of injected dose (ID) per gram of tissue (ID %/g) values were calculated with a formula [(gammacount observed/ID)×100]/weight of the tissue (g). The resulting values were plotted in a biodistribution graph presented in FIG. 14. The tracer [¹⁸F]25 demonstrated accumulation into tumor and a prolonged blood circulation time, with main elimination via kidney, but also demonstrated some liver uptake. The bone uptake was noticeably low, indicating the tracer was extremely stable towards defluorination in vivo.

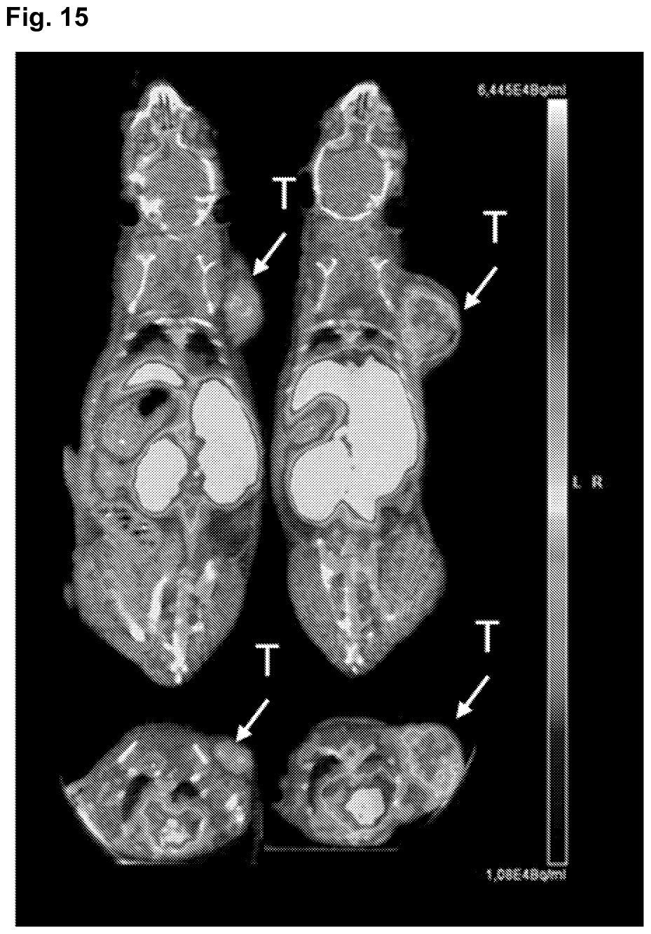

### Example 18. PET/CT of [¹⁸F]AmBF₃-Tyr³-Octreotide ([¹⁸F]25) in AR42J-Tumor Bearing Mice

[¹⁸F]AmBF₃-Tyr³-octreotide ([¹⁸F]25, 0.2 nmol, 150 μL, ˜1 MBq) was formulated in 4% ethanol in 0.01 M PBS and administered intravenously to AR42J tumor bearing Rj:NMRI-Foxn1 nu/nu mice. For investigating the specificity of the uptake in the AR42J tumors, mice were co-administered with blocking octreotide (44 nmol) for blocking the accumulation of radioactivity. The PET/CT image was acquired with Molecubes PET and CT. In the PEC/CT (FIG. 15) the tracer ([¹⁸F]25) demonstrated excellent stability proven by the lack of bone uptake. The main elimination route for the tracer was through kidneys into urine, but some minute accumulation was detected in the liver, gallbladder and intestines accounting for some background radioactivity levels in the PET image. First animal (the animal on the left in FIG. 15) was co-administered with blocking octreotide and radiolabeled [¹⁸F]25 intravenously. Second animal (the animal on the right in FIG. 15) received only [¹⁸F]25 without the blocking octreotide (45 μg, 44 nmol), enabling the visualization of the subcutaneous tumor on the right shoulder. The blocking of the radiolabeled [¹⁸F]25 was successful, demonstrating a specific uptake of radioactivity in the tumor, as seen in the PET image comparison of FIG. 15 (T=tumor).

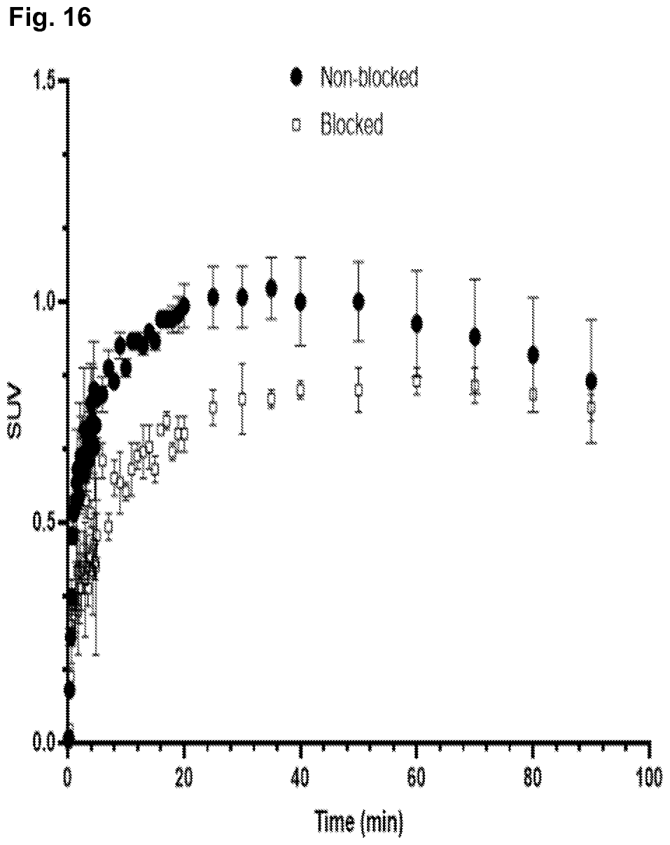

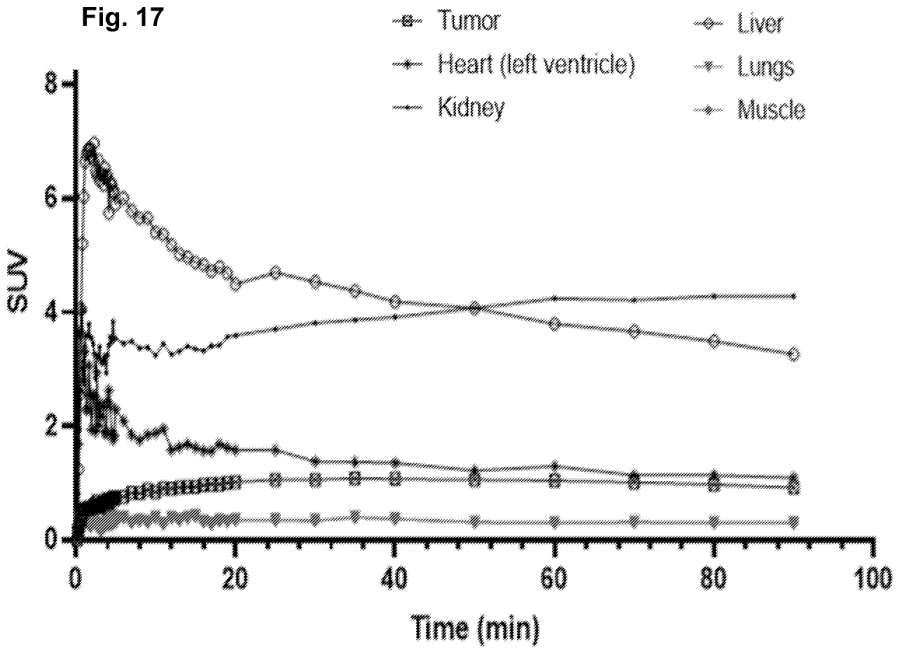

### Example 19. Elimination of Radioactivity after Intravenous Administration of [¹⁸F]AmBF₃-Tyr³-Octreotide ([¹⁸F]25) in AR42J Tumor Bearing Mice

[¹⁸F]AmBF₃-Tyr³-octreotide ([¹⁸F]25, 0.2 nmol, 150 μL, ˜1 MBq) was formulated in 4% ethanol in 0.01 M PBS and administered intravenously to AR42J tumor bearing Rj:NMRI-Foxn1 nu/nu mice. The standardized uptake values (SUVs) presenting the elimination profile (FIGS. 16 and 17) were determined from the PET image (FIG. 15) by ROIs around selected organs (heart, liver, kidney, lung, muscle, urinary bladder, tumor) and SUVs were calculated as disclosed in the example 16. The binding specificity of the [¹⁸F]25 in AR42J tumor bearing mice (n=2/group) was determined by comparing the SUV in tumor at different time points after mice were administered with [¹⁸F]25 alone (non-blocked) or co-administered with [¹⁸F]25 and blocking octreotide (Blocked) intravenously. The results demonstrate the binding of [¹⁸F]25 in AR42J tumor is specific and can be blocked by simultaneous administration of blocking octreotide (Blocked) (FIG. 16). The tracer ([¹⁸F]25) eliminated mainly through kidneys, with only minute accumulation in the liver that declined as a function of time (FIG. 17). The radioactivity in tumor peaked at around 40 minutes and remained relatively steady onwards. The blocking of the radioactivity in the tumor was demonstrated successfully in the SUV comparison data (FIG. 17), proving the tumor uptake was specific to the targeting moiety.

### Example 20. In Vitro Cell Internalization of [¹⁸F]AmBF₃—PSMA ([¹⁸F]28) and [¹⁸F]AmBF₃-Tranexamic Acid-PSMA ([¹⁸F]29)

C4-2 or LNCaP cells were plated 24 h prior the experiment on a 6 well plate (6×10⁵/well) and medium was changed to CO₂ independent medium 30 min. prior the experiment. The cells in each well were incubated with 1 mL of a 250 nM solution of [¹⁸F]28 or [¹⁸F]29 (15-20 GBq/mmol) CO₂ independent medium. Specific cellular uptake was determined by blocking with 2-(phosphonomethyl)pentanedioic acid (2-PMPA) (final concentration, 400 μM, Sigma). All experiments were conducted at 37° C. The incubation was terminated after 30, 60 and 120 min by washing 2 times with 1 mL of ice-cold phosphate-buffered saline. The cells were subsequently incubated twice with 1 mL of glycine HCl buffer (50 mM; pH 2.8) for 5 min each to remove the surface-bound fraction, and the supernatant was collected. After an additional washing step with 1 mL of ice-cold phosphate-buffered saline, the cells were lysed with 0.5 mL of NaOH (1 N) and collected, and radioactivity was measured with a γ-counter. Specific cellular uptake was calculated as a percentage of the initially added radioactivity bound to 10⁵ cells (% IA/10⁵ cells). All experiments were conducted in triplicates. Based on the results of this experiment, uptake of the tracers [¹⁸F]28 and [¹⁸F]29 into cells is specific, as it was blocked significantly (2.05±0.28% vs. 0.58±0.09% for [¹⁸F]28 and 1.05±0.09% vs. 0.31±0.03% for [¹⁸F]29) at 60 min, when the uptake was challenged with 2-PMPA blocking.

### Example 21. PET/CT of [¹⁸F]AmBF₃—PSMA ([¹⁸F]28) and [¹⁸F]AmBF₃-Tranexamic Acid-PSMA ([¹⁸F]29)

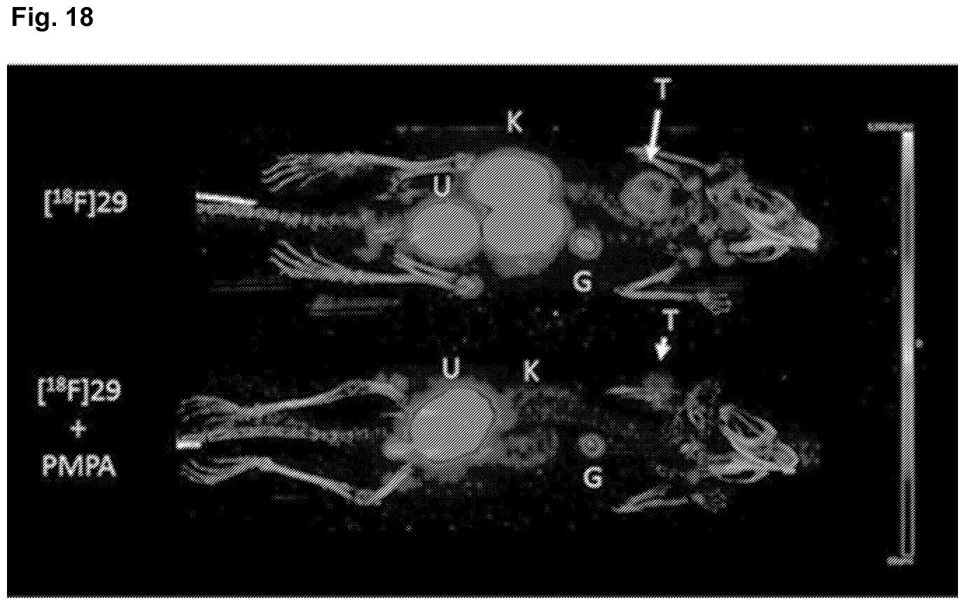

PET/CT imaging of [¹⁸F]28 or [¹⁸F]29 was conducted in C4-2 tumor bearing mice (n=3-4) and specificity of uptake was challenged by blocking with 2-PMPA. For blocking, the mice were injected with 0.4 mM 2-PMPA (100 μL; 40 nmol) via tail vein injection 30 min before injection of [¹⁸F]28 or [¹⁸F]29. The radiotracer, [¹⁸F]28 or [¹⁸F]29, was administered as a 0.01 mM solution (100 μL; 1 nmol) via tail vein injection. The PET/CT image was acquired with Inveon PET/CT and Molecubes PET and CT. In the PET/CT both tracers ([¹⁸F]28 and [¹⁸F]29) demonstrated from good to excellent stability proven by the lack of bone uptake. The main elimination route for the tracer was through kidneys into urine. Specific tumor uptake was demonstrated by blocking the uptake with 2-PMPA where tracer uptake was significantly reduced in tumor (T) as shown in the FIG. 18 for [¹⁸F]29.

### Example 22. Biodistribution of [¹⁸F]AmBF₃—PSMA ([¹⁸F]28) and [¹⁸F]AmBF₃-Tranexamic Acid-PSMA ([¹⁸F]29)

Biodistribution of [¹⁸F]28 or [¹⁸F]29 was determined in C4-2 tumor bearing mice and specificity of uptake was challenged by blocking with 2-PMPA. Each experiment was conducted in triplicates. For blocking, the SCID mice were administered with 0.4 mM 2-PMPA (100 μL; 40 nmol) via tail vein 30 min before injection of [¹⁸F]28 or [¹⁸F]29.

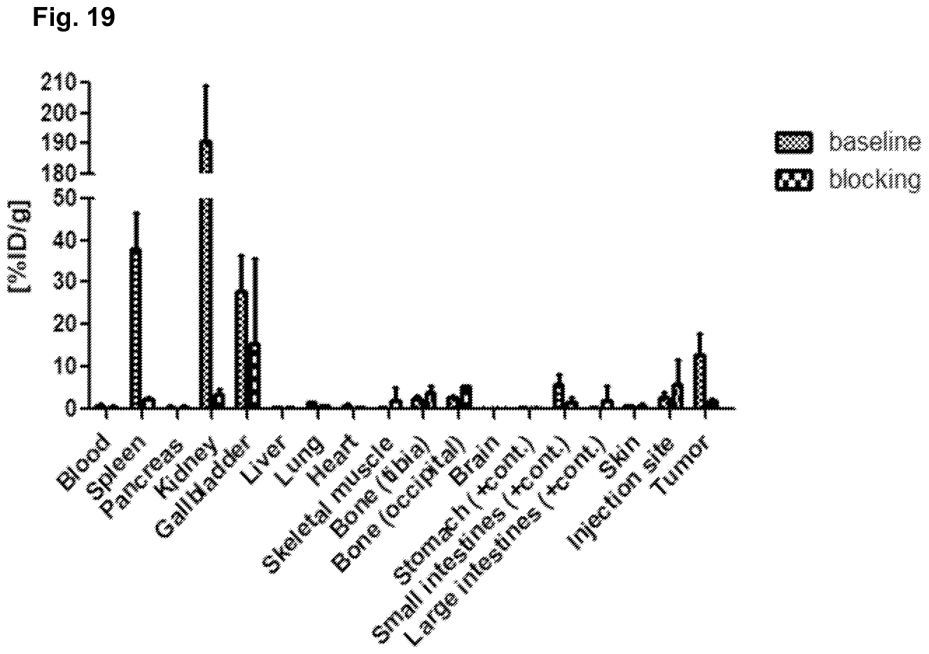

The radiotracer, [¹⁸F]28 or [¹⁸F]29, was administered as a 0.01 mM solution (100 μL; 1 nmol) via tail vein injection. At 1 h after injection, the animals were sacrificed (CO₂ asphyxiation), and organs of interest were dissected, blotted dry, and weighed. Radioactivity was measured with a γ-counter (1480 Wizard, PerkinElmer) and calculated as the percentage injected dose per gram (% ID/g). Tumor associated uptake for tracers [¹⁸F]28 or [¹⁸F]29 (FIG. 19) was blocked by pre-injection of 2-PMPA (7.51±0.69% vs. 1.18±0.19% for [¹⁸F]28 and 12.87±4.83% vs. 1.90±0.66% for [¹⁸F]29). Other organs experiencing high uptake values were kidneys, spleen and gallbladder, wherein uptake also decreased by application of the blocking agent.

### Example 23. Shelf Live and Plasma Stability of [¹⁸F]AmBF₃—PSMA ([¹⁸F]28), [¹⁸F]AmBF₃-Tranexamic Acid-PSMA ([¹⁸F]29)

To determine the shelf-life stability of [¹⁸F]28 or [¹⁸F]29 formulated in 1×PBS, a 5 μL sample was analyzed by radio-TLC after storing at room temp. for 0.5, 1, 2, 3, 4, 5 and 6 h (n=3). Stability (>95%) in PBS was proven for [¹⁸F]28 and [¹⁸F]29 for up to 6 h. For the determination of enzymatic stability, 400 μL of human plasma was incubated with 400 μL of the [¹⁸F]28 or [¹⁸F]29 formulation at 37° C. (n=3). After 0.5, 1, 2, 3 and 4 h, samples of 100 mL were removed from the mixture, the proteins were precipitated by addition of 50 μL of acetonitrile and separated from the supernatant by centrifugation at 13,000 rpm. The supernatant was analyzed by radio-TLC. Stability (>95%) in plasma was proven for [¹⁸F]28 and [¹⁸F]29 for up to 4 h. The compounds [¹⁸F]28 and [¹⁸F]29 did not show any significant degradation over the entire observation period of the experiment, both in formulated solution and in human plasma.

The foregoing description has provided by way of non-limiting examples of particular implementations and embodiments a full and informative description of the best mode presently contemplated by the inventors for carrying out the invention. It is however clear to a person skilled in the art that the invention is not restricted to details of the embodiments presented in the foregoing, but that it can be implemented in other embodiments using equivalent means or in different combinations of embodiments without deviating from the characteristics of the invention.

Furthermore, some of the features of the afore-disclosed example embodiments may be used to advantage without the corresponding use of other features. As such, the foregoing description shall be considered as merely illustrative of the principles of the present invention, and not in limitation thereof. Hence, the scope of the invention is only restricted by the appended patent claims.

Different non-binding example aspects and embodiments have been illustrated in the foregoing. The embodiments in the foregoing are used merely to explain selected aspects or steps that may be utilized in different implementations. Some embodiments may be presented only with reference to certain example aspects. It should be appreciated that corresponding embodiments may apply to other example aspects as well.

1. A tracer compound of formula (I), or a pharmaceutically acceptable salt or solvate thereof, 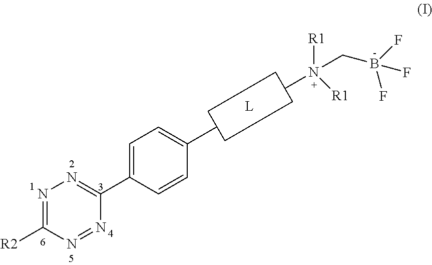
wherein:
each R1 is independently hydrogen (H) or an alkyl substituent with a formula CₙH₂ₙ₊₁, wherein n is an integer selected from the range 0-2; and
L is a linker moiety comprised of S1-Y—S2, wherein:
Y is (—CH₂—)ₘ wherein m is an integer selected from the range 1-4, or Y is a polyethylene glycol linker -(PEG)ₓ-, wherein (PEG)ₓ contains x repetitive units of polyethylene oxide —CH₂—CH₂—O— groups, and x is an integer selected from the range 1-20; and
S1 is —(CH₂)_{z}—CO—NH—(CH₂)_{z}—, or S1 is —(CH₂)_{z}—NH—CO—(CH₂)_{z}—, wherein each z is independently an integer selected from the range 0-4; and
S2 is —CH₂—, or S2 is —(CH₂)_{f}—CO—NH—(CH₂)_{f}—, or S2 is —(CH₂)_{f}—NH—CO—(CH₂)_{f}—, wherein each f is independently an integer selected from the range 0-4; and
R2 is either hydrogen (H) or a phenyl substituent or an alkyl substituent with a formula CₛH₂ₛ₊₁, wherein s is an integer selected from the range 0-2.

2. The tracer compound of claim 1, or a pharmaceutically acceptable salt or solvate thereof, wherein:
each R1 is independently hydrogen (H) or an alkyl substituent with a formula CₙH₂ₙ₊₁, wherein n is an integer selected from the range 0-2; and
L is a linker moiety comprised of S1-Y—S2, wherein:
Y is (—CH₂—)ₘ wherein m is an integer selected from the range 1-4, S1 is —CH₂—CO—NH—, or —CH₂—NH—CO— and S2 is —CH₂—; or
Y is a polyethylene glycol linker -(PEG)ₓ-, wherein (PEG)ₓ contains x repetitive units of polyethylene oxide —CH₂—CH₂—O— groups, and x is an integer selected from the range 1-20; and
S1 is —CH₂—NH—CO—(CH₂)_{z}—, or —CH₂—CO—NH—(CH₂)_{z}—, wherein each z is independently an integer selected from the range 0-4; and
S2 is —(CH₂)_{f}—CO—NH—CH₂—CH₂— or —(CH₂)_{f}—NH—CO—CH₂—CH₂—, wherein each f is independently an integer selected from the range 0-4; and
R2 is either hydrogen (H) or a phenyl substituent or an alkyl substituent with a formula CₛH₂ₛ₊₁, wherein s is an integer selected from the range 0-2.

3. The tracer compound of claim 1 or 2, wherein the x in -(PEG)ₓ- is an integer selected from the range 1-15 or from the range 1-10.

4. The tracer compound of one of the claims 1-3, wherein at least one F in the (BF₃)-moiety is a ¹⁸F.

5. An adduct of the tracer compound of one of the claims 1-4 and a -cyclooctene (TCO)-derivatized targeting moiety, or a pharmaceutically acceptable salt or solvate thereof, obtained through inverse electron demand Diels-Alder reaction (IEDDA) of a TCO moiety of the TCO-derivatized targeting moiety, with a tetrazine moiety of the tracer compound.

6. The adduct of claim 5, wherein a tetrazine ring of the tetrazine moiety of the tracer compound is chemically bound to the TCO-moiety of the TCO-derivatized targeting moiety.

7. The adduct of claim 5 or 6, wherein the targeting moiety is a protein, a peptide, an antibody, an antibody fragment, or a nanoparticle.

8. The adduct of one of the claims 5-7, wherein at least one F in the (BF₃)⁻ moiety is a ¹⁸F.

9. The adduct of one of the claims 5-8 for use in the detection of targeted entities in a subject by radioimaging, preferably by positron emission tomography imaging.

10. A method for manufacturing the tracer compound of one of the claims 1-3 comprising:
a. dissolving starting material into polar aprotic solvent and reacting the starting material with 2-(iodomethyl)-4,4,5,5-tetramethyl-1,3,2-dioxaborolane to provide an intermediate product; and
b. dissolving the intermediate product in polar aprotic solvent and reacting the intermediate product with KHF₂ in the presence of an acid such as HCl, water and organic solvent, to provide the tracer compound;
wherein the starting material is comprised of the tetrazine moiety linked via the linker moiety to a tertiary amine (—N(CH₃)₂); wherein
the tetrazine moiety is comprised of a 1,2,4,5-tetrazine ring, a phenyl ring attached to the C3 of the tetrazine ring, and R2 attached to the C6 of the tetrazine ring, wherein R2 is either hydrogen (H) or a phenyl substituent or an alkyl substituent with a formula CₛH₂ₛ+1, and s is an integer selected from the range 0-2; and
the linker moiety is comprised of S1-Y—S2, wherein:
Y is (—CH₂—)ₘ wherein m is an integer selected from the range 1-4, or Y is a polyethylene glycol linker -(PEG)ₓ-, wherein (PEG)ₓ contains x repetitive units of polyethylene oxide —CH₂—CH₂—O— groups, and x is an integer selected from the range 1-20; and
S1 is —(CH₂)_{z}—CO—NH—(CH₂)_{z}—, or S1 is —(CH₂)_{z}—NH—CO—(CH₂)_{z}—, wherein each z is independently an integer selected from the range 0-4; and
S2 is —CH₂—, or S2 is —(CH₂)_{f}—CO—NH—(CH₂)_{f}—, or S2 is —(CH₂)_{f}—NH—CO—(CH₂)_{f}—, wherein each f is independently an integer selected from the range 0-4.

11. The method of claim 10, wherein the linker moiety of the starting material is comprised of S1-Y—S2, wherein:
Y is (—CH₂—)ₘ wherein m is an integer selected from the range 1-4, S1 is —CH₂—CO—NH—, or —CH₂—NH—CO— and S2 is —CH₂—; or
Y is a polyethylene glycol linker -(PEG)ₓ-, wherein (PEG)ₓ contains x repetitive units of polyethylene oxide —CH₂—CH₂—O— groups, and x is an integer selected from the range 1-20; and
S1 is —CH₂—NH—CO—(CH₂)_{z}—, or —CH₂—CO—NH—(CH₂)_{z}—, wherein each z is independently an integer selected from the range 0-4; and
S2 is —(CH₂)_{f}—CO—NH—CH₂—CH₂— or —(CH₂)_{f}—NH—CO—CH₂—CH₂—, wherein each f is independently an integer selected from the range 0-4.

12. A method for manufacturing the adduct of one of the claims 5-8 or a pharmaceutically acceptable salt or solvate thereof, the method comprising:
providing the TCO-derivatized targeting moiety;
providing the tracer compound of any of the claims 1-3; and
allowing the tetrazine moiety of the tracer compound to react with the TCO moiety of the TCO-derivatized targeting moiety; and
radiolabeling the adduct with at least one ¹⁸F to obtain the adduct;
or the method comprising:
providing the TCO-derivatized targeting moiety;
providing the tracer compound of any of the claims 1-3;
radiolabeling the tracer compound of any of the claims 1-3 with at least one ¹⁸F; and
allowing the tetrazine moiety of the radiolabeled tracer compound to react with the TCO moiety of the TCO-derivatized targeting moiety to obtain the adduct.

13. The method of claim 12, comprising:
allowing the tetrazine moiety of the radiolabeled tracer compound to react with the TCO moiety of the TCO-derivatized targeting moiety at a temperature between 20-80° C., preferably at a temperature between 40-70° C., more preferably at a temperature between 55-65° C., most preferably at a temperature 60° C.

14. Use of the tracer compound of the claims 1-4 and/or the adduct of the claims 5-8 in detection of targeted entities in a subject by radioimaging the subject, wherein the entities are targeted with the radiolabeled tracer compounds and/or the adducts.

15. A kit for production of the ¹⁸F-labeled adduct for detection of targeted entities in a subject with radioimaging, comprising at least one compartment containing the tracer compound of claims 1-3, at least one compartment containing at least one TCO-derivatized targeting moiety, at least one compartment containing ¹⁸F for radiolabeling the tracer compound, and optionally aqueous and organic solvents for the IEDDA conjugation and radiolabeling of the tracer compound and/or the adduct.
